(19) United States
(12) Patent Application Publication
(10) Pub. No.: US 2025/0017941 A1
(43) Pub. Date: Jan. 16, 2025

(54) METHODS OF TREATING NON-HODGKIN LYMPHOMA USING 2-(2,6-DIOXOPIPERIDIN-3-YL)-4-((2-FLUORO-4-((3-MORPHOLINOAZETIDIN-1-YL)METHYL)BENZYL)AMINO)ISOINDOLINE-1,3-DIONE

(71) Applicants: Celgene Corporation, Summit, NJ (US); Bristol-Myers Squibb Company, Princeton, NJ (US)

(72) Inventors: Tonia J. Buchholz, Moss Beach, CA (US); Michael Pourdehnad, San Francisco, CA (US); Poliana Alves Patah, Princeton, NJ (US); Fan Wu, Livingston, NJ (US)

(21) Appl. No.: 18/901,764

(22) Filed: Sep. 30, 2024

Related U.S. Application Data
(63) Continuation of application No. 17/725,398, filed on Apr. 20, 2022.
(60) Provisional application No. 63/177,616, filed on Apr. 21, 2021.

(51) Int. Cl. A61K 31/5377; A61K 45/06

(52) U.S. Cl. CPC A61K 31/5377; A61K 45/06

(57) ABSTRACT

Provided herein are methods of using (S)-2-(2,6-dioxopiperidin-3-yl)-4-((2-fluoro-4-((3- morpholinoazetidin-1-yl)methyl)benzyl)amino)isoindoline-1,3-dione, or an enantiomer, a mixture of enantiomers, a tautomer, an isotopolog, or a pharmaceutically acceptable salt thereof, in combination with tafasitamab, obinutuzumab, or tazemetostat, for treating, preventing or managing non-Hodgkin lymphoma.

This application is a continuation of U.S. patent application Ser. No. 17/725,398, filed on Apr. 20, 2022, which claims priority to U.S. Provisional Application No. 63/177,616, filed on Apr. 21, 2021, the entirety of each of which is incorporated herein by reference.

### FIELD

Provided herein are methods of using 2-(2,6-dioxopiperidin-3-yl)-4-((2-fluoro-4-((3-morpholinoazetidin-1-yl)methyl)benzyl)amino) isoindoline-1,3-dione, or an enantiomer, a mixture of enantiomers, a tautomer, an isotopolog, or a pharmaceutically acceptable salt thereof, in combination with tafasitamab, obinutuzumab, or tazemetostat, for treating, preventing or managing non-Hodgkin lymphoma.

### BACKGROUND

Cancer is characterized primarily by an increase in the number of abnormal cells derived from a given normal tissue, invasion of adjacent tissues by these abnormal cells, or lymphatic or blood-borne spread of malignant cells to regional lymph nodes and metastasis. Clinical data and molecular biologic studies indicate that cancer is a multistep process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. Current cancer therapy may involve surgery, chemotherapy, hormonal therapy and/or radiation treatment to eradicate neoplastic cells in a patient. Recent advances in cancer therapeutics are discussed by Rajkumar et al. in Nature Reviews Clinical Oncology 11, 628-630 (2014).

All of the current cancer therapy approaches pose significant drawbacks for the patient. Surgery, for example, may be contraindicated due to the health of a patient or may be unacceptable to the patient. Additionally, surgery may not completely remove neoplastic tissue. Radiation therapy is only effective when the neoplastic tissue exhibits a higher sensitivity to radiation than normal tissue. Radiation therapy can also often elicit serious side effects. Hormonal therapy is rarely given as a single agent. Although hormonal therapy can be effective, it is often used to prevent or delay recurrence of cancer after other treatments have removed the majority of cancer cells.

With respect to chemotherapy, there are a variety of chemotherapeutic agents available for treatment of cancer. A majority of cancer chemotherapeutics act by inhibiting DNA synthesis, either directly or indirectly by inhibiting the biosynthesis of deoxyribonucleotide triphosphate precursors, to prevent DNA replication and concomitant cell division. Gilman et al., Goodman and Gilman's: The Pharmacological Basis of Therapeutics, Tenth Ed. (McGraw Hill, New York).

Despite availability of a variety of chemotherapeutic agents, chemotherapy has many drawbacks. Stockdale, Medicine, vol. 3, Rubenstein and Federman, eds., ch. 12, sect. 10, 1998. Almost all chemotherapeutic agents are toxic, and chemotherapy causes significant, and often dangerous side effects including severe nausea, bone marrow depression, and immunosuppression. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even if those agents act by different mechanism from those of the drugs used in the specific treatment. This phenomenon is referred to as pleiotropic drug or multidrug resistance. Because of the drug resistance, many cancers prove or become refractory to standard chemotherapeutic treatment protocols.

Non-Hodgkin lymphoma (NHL), also known as non-Hodgkin's lymphoma, is the fifth most common cancer for both men and women in the United States. An estimated 385,700 patients worldwide were diagnosed with NHL in 2012 and approximately 199,700 patients died as a result of the disease. Torre, L. A. et al. Global cancer statistics, 2012; CA Cancer J. Clin. 65, 87-108 (2015). NHL is a heterogeneous disease comprising diverse B-cell and T-cell lymphoma subtypes that collectively make up approximately 4% of all new cancer cases in the United States (U.S.) and account for 3% of cancer-related deaths. Most of NHLs (80% to 90%) are of B-cell origin, and the great majority of the rest are T-cell lymphomas. Common subtypes of NHL include diffuse large B-cell lymphoma (DLBCL), follicular lymphoma (FL), mantle cell lymphoma (MCL), and primary central nervous system lymphoma (PCNSL).

Diffuse large B-cell lymphoma (DLBCL) is the most common subtype of NHL, accounting for up to 30% of newly diagnosed cases, and is clinically classified as an aggressive lymphoma. With the introduction of rituximab plus chemotherapy combination regimens, more than 50% of patients with DLBCL are cured. However, more than 30% of patients in remission will ultimately relapse. For patients who relapse, treatment approaches for second-line DLBCL are less well defined and often are ineffective in achieving long-term disease control. In patients who have received 2 or more lines of therapy and are relapsed and/or refractory and are not candidates for potentially curative therapies due to advanced age or poor performance status, DLBCL remains an incurable disease for which clinical trials are indicated. New therapeutic approaches are still needed.

For follicular lymphoma (FL), the age adjusted incidence rate from 2011-2012 in the U.S. was 3.4 per 100,000. There is no standard treatment for relapsed or refractory (R/R) FL patients. Despite the efforts and advances in front-line treatment, patients with FL continue to experience recurring relapses and require further therapy. The first-line systemic anti-cancer treatments are also considered in second-line therapy; more recently, second-line or later therapy options may include “chemotherapy-free” regimens that are being developed and may become standard of care in the near future. In third-line, patients who fail to respond to a rituximab-containing regimen and have relapsed on or are refractory to additional therapy have limited treatment options and a poor prognosis. There is a high unmet medical need to develop novel treatments for FL patients who fail to respond to standard therapy and whose treatment options for disease remission have been exhausted.

Approximately 6% of all new lymphoma cases each year are mantle cell lymphoma (MCL). The age adjusted incidence rate from 2011-2012 in the U.S. for mantle cell lymphoma (MCL) was 0.8 per 100,000. Despite several available frontline therapies for MCL with prolonged responses, MCL remains an incurable B-cell malignancy. Patients with MCL are often treated with rituximab-chemotherapy combinations, either with or without stem cell transplant consolidation. Relapse is typical, and MCL becomes increasingly resistant to therapy over time.

The age adjusted incidence rate from 2011-2012 in the U.S. for primary central nervous system lymphoma (PCNSL) was 0.3 per 100,000. Despite high response rates with initial high-dose methotrexate (HD-MTX)-based regimens, more than half of the responders relapse. Once PCNSL has relapsed, prognosis remains poor. Novel therapeutic agents with CNS penetration, better efficacy, and tolerable toxicity profile are urgently needed.

There remains a significant need for safe and effective methods of treating, preventing and managing NHL, particularly for NHL that is refractory to standard treatments, such as surgery, radiation therapy, chemotherapy and hormonal therapy, while reducing or avoiding the toxicities and/or side effects associated with conventional therapies.

Citation or identification of any reference in this section of this application is not to be construed as an admission that the reference is prior art to the present application.

### SUMMARY

Provided herein are methods of using (S)-2-(2,6-dioxopiperidin-3-yl)-4-((2-fluoro-4-((3-morpholinoazetidin-1-yl)methyl)benzyl)amino)isoindoline-1,3-dione, or an enantiomer, a mixture of enantiomers, a tautomer, an isotopolog, or a pharmaceutically acceptable salt thereof, in combination with a second therapeutic agent, for treating, preventing or managing NHL. The second therapeutic agent is tafasitamab, obinutuzumab, or tazemetostat.

In certain embodiments, provided herein is a method of treating NHL, comprising administering to a subject in need thereof a therapeutically effective amount of a compound of Formula (I):

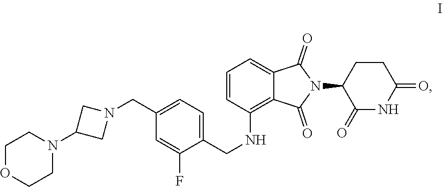 [SMILES: O=C1CC[C@H](N2C(=O)c3cccc(NCc4ccc(CN5CC(N6CCOCC6)C5)cc4F)c3C2=O)C(=O)N1]

or an enantiomer, mixture of enantiomers, tautomer, isotopolog, or pharmaceutically acceptable salt thereof, in combination with a second therapeutic agent, wherein the second therapeutic agent is tafasitamab, obinutuzumab, or tazemetostat.

In certain embodiments, provided herein is a method of treating NHL, comprising administering to a subject in need thereof a therapeutically effective amount of a hydrochloride salt of a compound of Formula (I), in combination with a second agent provided herein.

In certain embodiments, the NHL is diffuse large B-cell lymphoma (DLBCL), follicular lymphoma (FL), mantle cell lymphoma (MCL), or primary central nervous system lymphoma (PCNSL). In certain embodiments, the NHL is relapsed or refractory NHL.

The present embodiments can be understood more fully by reference to the detailed description and examples, which are intended to exemplify non-limiting embodiments.

### DETAILED DESCRIPTION

### Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein, and in the specification and the accompanying claims, the indefinite articles “a” and “an” and the definite article “the” include plural as well as single referents, unless the context clearly indicates otherwise.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of a compound provided herein include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments provided herein, including mixtures thereof.

The use of stereomerically pure forms of such compounds, as well as the use of mixtures of those forms, are encompassed by the embodiments provided herein. For example, mixtures comprising equal or unequal amounts of the enantiomers of a particular compound may be used in methods and compositions provided herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuja, S., Chiral Separation Methods for Pharmaceutical and Biotechnological Products (John Wiley & Sons, 2011).

It is to be understood that the compounds provided herein may contain chiral centers. Such chiral centers may be of either the (R) or(S) configuration, or may be a mixture thereof. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its(S) form.

Optically active (+) and (−), (R)- and (S)-, or (D)- and (L)-isomers may be prepared using chiral synthons or chiral reagents, or resolved using conventional techniques, such as chromatography on a chiral stationary phase.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

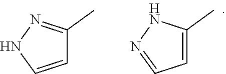

As readily understood by one skilled in the art, a wide variety of functional groups and other structures may exhibit tautomerism and all tautomers of a compound are within the scope of the compound as provided herein.

It should also be noted that a compound provided herein can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of a compound, whether radioactive or not, are intended to be encompassed within the scope of the compound as provided herein. In some embodiments, provided herein are isotopologues of the compounds, for example, the isotopologues are deuterium, carbon-13 (¹³C), and/or nitrogen-15 (¹⁵N) enriched compounds. As used herein, “deuterated”, means a compound wherein at least one hydrogen (H) has been replaced by deuterium (indicated by D or ²H), that is, the compound is enriched in deuterium in at least one position.

It is understood that, independently of stereomerical or isotopic composition, each compound provided herein can be provided in the form of any of the pharmaceutically acceptable salts provided herein. Equally, it is understood that the isotopic composition may vary independently from the stereomerical composition of each compound provided herein. Further, the isotopic composition, while being restricted to those elements present in the respective compound or salt thereof, may otherwise vary independently from the selection of the pharmaceutically acceptable salt of the respective compound.

It should be noted that if there is a discrepancy between a depicted structure and a name for that structure, the depicted structure is to be accorded more weight.

As used herein and unless otherwise indicated, the term “treating” means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself.

As used herein and unless otherwise indicated, the term “preventing” means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition.

As used herein and unless otherwise indicated, the term “managing” encompasses preventing the recurrence of the particular disease or disorder in a patient who had suffered from it, lengthening the time a patient who had suffered from the disease or disorder remains in remission, reducing mortality rates of the patients, and/or maintaining a reduction in severity or avoidance of a symptom associated with the disease or condition being managed.

As used herein and unless otherwise indicated, the term “effective amount” in connection with a compound means an amount capable of treating, preventing, or managing a disorder, disease or condition, or symptoms thereof.

As used herein and unless otherwise indicated, the term “subject” includes an animal, including, but not limited to, an animal such a cow, monkey, horse, sheep, pig, chicken, turkey, quail, cat, dog, mouse, rat, rabbit or guinea pig, in one embodiment a mammal, in another embodiment a human.

As used herein and unless otherwise indicated, the term “fed” means a subject starts a meal about 10 minutes, about 15 minutes, about 20 minutes, about 25 minutes, about 30 minutes, about 40 minutes, about 45 minutes, about 50 minutes or about 1 hour prior to the administration of a compound provided herein. In one embodiment, a compound provided herein is administered when the subject started a meal 30 minutes prior to such administration.

As used herein and unless otherwise indicated, the term “fasted” means a subject has no food intake for at least about 5 hours, about 6 hours, about 7 hours, about 8 hours, about 9 hours, about 10 hours, about 11 hours, about 12 hours or has an overnight fast. In one embodiment, a compound provided herein is administered after a subject had no food intake for about 8 hours. In one embodiment, a compound provided herein is administered after a subject had no food intake for at least about 8 hours. In one embodiment, a compound provided herein is administered after a subject had an overnight fast of at least about 8 hours. In one embodiment, a compound provided herein is administered after a subject had no food intake for about 10 hours. In one embodiment, a compound provided herein is administered after a subject had no food intake for at least about 10 hours. In one embodiment, a compound provided herein is administered after a subject had an overnight fast of at least about 10 hours.

As used herein and unless otherwise indicated, the term “relapsed” refers to a disorder, disease, or condition that responded to treatment (e.g., achieved a complete response) then had progression. The treatment can include one or more lines of therapy.

In one embodiment, “relapsed” DLBCL may refer to DLBCL that has been previously treated with one or more lines of therapy. In one embodiment, the relapsed DLBCL is DLBCL that has been previously treated with one, two, three or four lines of therapy. In one embodiment, the relapsed DLBCL is DLBCL that has been previously treated with two or more lines of treatment.

In one embodiment, “relapsed” FL may refer to FL that has been previously treated with one or more lines of therapy. In one embodiment, the relapsed FL is FL that has been previously treated with one, two, three or four lines of therapy. In one embodiment, the relapsed FL is FL that has been previously treated with two or more lines of treatment.

In one embodiment, “relapsed” MCL may refer to MCL that has been previously treated with one or more lines of therapy. In one embodiment, the relapsed MCL is MCL that has been previously treated with one, two, three or four lines of therapy. In one embodiment, the relapsed MCL is MCL that has been previously treated with two or more lines of treatment.

In one embodiment, “relapsed” PCNSL may refer to PCNSL that has been previously treated with one or more lines of therapy. In one embodiment, the relapsed PCNSL is PCNSL that has been previously treated with one, two, three or four lines of therapy. In one embodiment, the relapsed PCNSL is PCNSL that has been previously treated with two or more lines of treatment.

As used herein and unless otherwise indicated, the term “refractory” refers to a disorder, disease, or condition that has not responded to prior treatment that can include one or more lines of therapy. In one embodiment, the disorder, disease, or condition has been previously treated one, two, three or four lines of therapy. In one embodiment, the disorder, disease, or condition has been previously treated with two or more lines of treatment, and has less than a complete response (CR) to most recent systemic therapy containing regimen.

In one embodiment, a relapsed or refractory DLBCL has been previously treated with at least one prior line of therapy. In one embodiment, a relapsed or refractory DLBCL has been previously treated with at least two prior lines of therapy. In one embodiment, no more than one of the prior lines of therapy may be a treatment for lower grade lymphoma. In one embodiment, a relapsed or refractory DLBCL has been previously treated with at least one standard treatment regimen for DLBCL. In one embodiment, a relapsed or refractory DLBCL has been previously treated with one or more systemic regimens, and wherein one or more of the systemic regimens comprise an anti-CD20 therapy. In certain embodiments, the subject has received one to three systemic regimens, and wherein at least one of the systemic regimens is an anti-CD20 therapy.

In one embodiment, a relapsed or refractory FL has been previously treated with at least one prior line of therapy. In one embodiment, a relapsed or refractory FL has been previously treated with at least two prior lines of therapy. In one embodiment, a relapsed or refractory FL is Grade 1, 2, 3a or 3b according to Groupe d'Etude des Lymphomes Folliculaires (GELF) criteria (National Comprehensive Cancer Network. NCCN Clinical Practice Guidelines in Oncology: B-cell Lymphomas; V.2.2018. 2018 Feb. 26; V.2, the entirety of which is incorporated herein by reference). In one embodiment, a relapsed or refractory FL has been previously treated with one or more systemic regimens, and wherein one or more of the systemic regimens comprise an anti-CD20 therapy. In certain embodiments, the subject has received one to three systemic regimens, and wherein at least one of the systemic regimens is an anti-CD20 therapy.

In one embodiment, a relapsed or refractory MCL has been previously treated with at least one prior line of therapy. In one embodiment, a relapsed or refractory MCL has been previously treated with at least two prior lines of therapy. In one embodiment, a relapsed or refractory MCL subject has progressed on or been refractory to at least one BTK inhibitor containing regimen. In one embodiment, the BTK inhibitor is ibrutinib. In one embodiment, the BTK inhibitor is acalabrutinib.

In one embodiment, a relapsed or refractory PCNSL has been previously treated with at least one prior line of therapy. In one embodiment, a relapsed or refractory PCNSL has been previously treated with at least two prior lines of therapy.

In the context of a cancer, inhibition may be assessed by inhibition of disease progression, inhibition of tumor growth, reduction of primary tumor, relief of tumor-related symptoms, inhibition of tumor secreted factors, delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others. OS as used herein means the time from treatment onset until death from any cause. TTP as used herein means the time from treatment onset until tumor progression; TTP does not include deaths. In one embodiment, PFS means the time from treatment onset until tumor progression or death. In one embodiment, PFS means the time from the first dose of compound to the first occurrence of disease progression or death from any cause. In one embodiment, PFS rates is computed using the Kaplan-Meier estimates. Event-free survival (EFS) means the time from treatment onset until any treatment failure, including disease progression, treatment discontinuation for any reason, or death. In one embodiment, overall response rate (ORR) means the percentage of patients who achieve a response. In one embodiment, ORR means the sum of the percentage of patients who achieve complete and partial responses. In one embodiment, ORR means the percentage of patients whose best response≥partial response (PR). In one embodiment, duration of response (DoR) is the time from achieving a response until relapse or disease progression. In one embodiment, DoR is the time from achieving a response≥partial response (PR) until relapse or disease progression. In one embodiment, DoR is the time from the first documentation of a response until to the first documentation of progressive disease or death. In one embodiment, DoR is the time from the first documentation of a response≥partial response (PR) until to the first documentation of progressive disease or death. In one embodiment, time to response (TTR) means the time from the first dose of compound to the first documentation of a response. In one embodiment, TTR means the time from the first dose of compound to the first documentation of a response≥partial response (PR). In the extreme, complete inhibition, is referred to herein as prevention or chemoprevention. In this context, the term “prevention” includes either preventing the onset of clinically evident cancer altogether or preventing the onset of a preclinically evident stage of a cancer. Also intended to be encompassed by this definition is the prevention of transformation into malignant cells or to arrest or reverse the progression of premalignant cells to malignant cells. This includes prophylactic treatment of those at risk of developing a cancer.

In certain embodiments, the treatment of NHL may be assessed by the International Workshop Criteria for Malignant Lymphoma (see Cheson et al., J. Clin. Oncol. 2014, 32(27):3059-3068) and the Deauville Criteria for fluorodeoxyglucose-positron emission tomography (FDG-PET) scan interpretation (Itti et al., Eur. J. Nucl. Med. Mol. Imaging, 2013, 40(9):1312-20; Meignan et al., Leuk Lymphoma, 2014, 55(1):31-37) (“Lugano criteria”), using the response and end point definition shown in Tables 1-3.

TABLE 1
Criteria for Involvement of Site.

| Tissue Site | Clinical | FDG Avidity | Test | Positive Finding |
|---|---|---|---|---|
| Lymph nodes | Palpable | FDG-avid histologies | PET/CT | Increase FDG uptake |
| | | Nonavid disease | CT | Unexplained node |
| | | | | enlargement |
| Spleen | Palpable | FDG-avid histologies | PET/CT | Diffuse uptake, solitary |
| | | | | mass, miliary lesions, |
| | | | | nodules |
| | | Nonavid disease | CT | >13 cm in vertical length, |
| | | | | mass, nodules |
| Liver | Palpable | FDG-avid histologies | PET/CT | Diffuse uptake, mass |
| | | Nonavid disease | CT | Nodules |
| CNS | Signs, | N/A | CT | Mass lesion(s) |
| | symptoms | | MRI | Leptomeningeal |
| | | | | infiltration, mass lesions |
| | | | CSF | Cytology, flow cytometry |
| | | | assessment | |
| Other (eg, skin, | Site dependent | N/A | PET/CT^{a}, | Lymphoma involvement |
| lung, GI tract, | | | biopsy | |
| bone, bone marrow) | | | | |

CNS = central nervous system; CSF = cerebrospinal fluid; CT = computed tomography; FDG = fluorodeoxyglucose; GI = gastrointestinal; MRI = magnetic resonance imaging; PET = positron emission tomography; N/A = not applicable.
^{a}PET/CT is adequate for determination of bone marrow involvement and can considered highly suggestive for involvement of other extralymphatic sites. Biopsy confirmation of those sites can be considered if necessary.

TABLE 2
Lugano Response Criteria for Non-Hodgkin Lymphoma.

| Response | Site | PET/CT (metabolic response) | CT (Radiologic response) |
|---|---|---|---|
| Complete | Lymph nodes | Score 1, 2, 3 with or without | All of the following: |
| response | and | residual mass on 5-PS (Table | Target nodes/nodal masses must |
| | extralymphatic | 3) | regress to ≤1.5 cm in LDi |
| | sites | | No extralymphatic sites of disease |
| | Non-measured | N/A | Absent |
| | lesion | | |
| | Organ | N/A | Regress to normal |
| | enlargement | | |
| | New Lesions | None | None |
| | Bone Marrow | No evidence of FDG-avid | Normal by morphology; if |
| | | disease in marrow | inderterminate, IHC negative^{a} |
| Partial | Lymph nodes | Score 4 or 5 on 5-PS with | All of the following: |
| Response | and | reduced uptake compared with | ≥50% decrease in SPD of up to 6 |
| | extralymphatic | baseline and residual mass(es) | target measureable nodes and |
| | sites | of any size | extranodal sites |
| | | At interim these findings | When a lesion is too small to measure |
| | | suggest responding disease | on CT, assign 5 mm × 5 mm as the |
| | | At end of treatment these | default value |
| | | findings may indicate residual | When no longer visible, 0 mm × 0 mm |
| | | disease | For a node >5 mm × 5 mm, but |
| | | | smaller than normal, use actual |
| | | | measurement for calculation |
| | Non-measured | N/A | Absent/normal, regressed, but no |
| | lesion | | increase |
| | Organ | N/A | Spleen must have regressed by >50% |
| | enlargement | | in length beyond normal |
| | New Lesions | None | None |
| | Bone Marrow | Residual uptake higher than | N/A |
| | | uptake in normal marrow but | |
| | | reduced compared with | |
| | | baseline. If persistent focal | |
| | | changes in the marrow in the | |
| | | context of nodal response, | |
| | | consider MRI or biopsy or | |
| | | interval scan | |
| Stable | Target | Score 4 or 5 on 5-PS with no | <50% decrease from baseline of up to |
| Disease | nodes/nodal | significant change in FDG | 6 dominant, measureable nodes and |
| | masses, | uptake from baseline | extranodal sites |
| | extranodal | | No criteria for progressive disease are |
| | lesions | | met |
| | Non-measured | N/A | No increase consistent with |
| | lesion | | progression |
| | Organ | N/A | No increase consistent with |
| | enlargement | | progression |
| | New Lesions | None | None |
| | Bone Marrow | No change from baseline | N/A |
| Progressive | Lymph nodes | Score 4 or 5 on 5-PS with an | At least one of the following: |
| Disease | and | increase in intensity of uptake | PPD progression: |
| | extralymphatic | compared with baseline | An individual node/lesion must be |
| | sites | and/or | abnormal with: |
| | | New FDG-avid foci consistent | LDi >1.5 cm and |
| | | with lymphoma | Increase by ≥50% from PPD nadir and |
| | | | An increase in LDi or SDi from nadir |
| | | | 0.5 cm for lesions ≤2 cm |
| | | | 1.0 cm for lesions >2 cm |
| | | | In the setting of splenomegaly, |
| | | | splenic length must increase by >50% |
| | | | of the extent of its prior increase |
| | | | above baseline (eg, a 15 cm spleen |
| | | | must increase to >16 cm). If no |
| | | | splenomegaly, must increase by at |
| | | | least 2 cm from baseline must |
| | | | increase by at least 2 cm from |
| | | | baseline |
| | | | New or recurrent splenomegaly |
| | Non-measured | None | New or clear progression of |
| | lesion | | preexisting nonmeasured lesions |
| | New Lesions | New FDG-avid foci consistent | Regrowth of previously resolved |
| | | with lymphoma rather than | lesions |
| | | another etiology (eg, infection, | A new node >1.5 cm in any axis |
| | | inflammation). If uncertain | A new extranodal site >1.0 cm in any |
| | | etiology, consider biopsy or | axis; if <1.0 cm in any axis, its |
| | | interval scan | presence must be unequivocal and |
| | | | must be attributable to lymphoma |
| | | | Assessable disease of any size |
| | | | unequivocally attributable to |
| | | | lymphoma |
| | Bone Marrow | New of recurrent FDG-avid | New or recurrent involvement |
| | | foci | |

CMR = complete metabolic response; LDi = longest transverse diameter of a lesion; PPD = cross product of the LDi and perpendicular diameter; SDi = shortest axis perpendicular to the LDi; SPD = sum of the product of the perpendicular diameters for multiple lesions; N/A = not applicable.
^{a}Required for CR if bone marrow involvement at baseline
^{b}In Waldeyer's ring or extranodal sites with high physiologic uptake or with activation within spleen or marrow; (eg with chemotherapy or myeloid colony stimulating factors), uptake may be greater than normal mediastinum and/or liver. In this circumstance, CMR may be inferred if uptake at sites of initial involvement is no greater than surrounding normal tissue.
^{c}FDG-avid lymphomas should have response assessed by PET-CT. Some diseases can typically be followed with CT alone (i.e., marginal zone lymphoma).
^{d}PET should be done with contrast-enhanced diagnostic CT and can be done simultaneously or at separate procedures.

TABLE 3
PET Five Point Scale (5-PS).

| | |
|---|---|
| 1 | No uptake above background |
| 2 | Uptake ≤ mediastinum |
| 3 | Uptake > mediastinum but ≤ liver |
| 4 | Uptake moderately > liver |
| 5 | Uptake markedly higher than liver and/or new lesions |
| X | New areas of uptake unlikely to be related to lymphoma |

^{a}The Deauville five-point scale (5PS) is an internationally recommended scale for clinical routine and clinical trials using FDG-PET/CT in the initial staging and assessment of treatment response in Hodgkin lymphoma (HL) and certain types of non-Hodgkin lymphomas (NHL).

In certain embodiments, stable disease or lack thereof can be determined by methods known in the art such as evaluation of patient symptoms, physical examination, visualization of the tumor that has been imaged, for example using FDG-PET (fluorodeoxyglucose positron emission tomography), PET/CT (positron emission tomography/computed tomography) scan, MRI (magnetic resonance imaging) Brain/Spine, CSF (cerebrospinal fluid), ophthalmologic exams, vitreal fluid sampling, retinal photograph, bone marrow evaluation and other commonly accepted evaluation modalities.

As used herein and unless otherwise indicated, the terms “co-administration” and “in combination with” include the administration of one or more therapeutic agents (for example, a compound provided herein and another anti-NHL agent, cancer agent or supportive care agent) either simultaneously, concurrently or sequentially with no specific time limits. In one embodiment, the agents are present in the cell or in the patient's body at the same time or exert their biological or therapeutic effect at the same time. In one embodiment, the therapeutic agents are in the same composition or unit dosage form. In another embodiment, the therapeutic agents are in separate compositions or unit dosage forms.

As used herein and unless otherwise specified, “a therapeutic agent” provided herein is not limited to a single therapeutic agent, and it can be, in certain embodiments, a combination of one or more different therapeutic agents. The one or more therapeutic agents can be administered in combination with each other as described herein. As used herein and unless otherwise specified, “a therapeutic agent” can be used interchangeably with “a therapeutic therapy”, and is not limited to a therapeutic substance. For example, a therapeutic agent can be a cancer treatment such as radiation therapy or CAR-T therapy.

The term “supportive care agent” refers to any substance that treats, prevents or manages an adverse effect from treatment with another therapeutic agent.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In one embodiment, the terms “about” and “approximately,” when used in this context, contemplate a dose, amount, or weight percent within 30%, within 20%, within 15%, within 10%, or within 5%, of the specified dose, amount, or weight percent.

### COMPOUNDS

In one embodiment, the compound used in the methods provided herein is (S)-2-(2,6-dioxopiperidin-3-yl)-4-((2-fluoro-4-((3-morpholinoazetidin-1-yl)methyl)benzyl)amino)isoindoline-1,3-dione of the following formula:

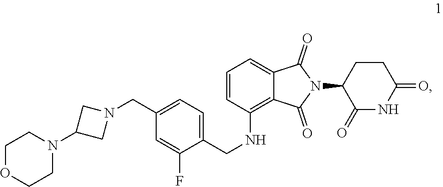 [SMILES: O=C1CC[C@H](N2C(=O)c3cccc(NCc4ccc(CN5CC(N6CCOCC6)C5)cc4F)c3C2=O)C(=O)N1]

or an enantiomer, mixture of enantiomers, tautomer, isotopolog, or pharmaceutically acceptable salt thereof. (S)-2-(2,6-dioxopiperidin-3-yl)-4-((2-fluoro-4-((3-morpholinoazetidin-1-yl)methyl)benzyl)amino)isoindoline-1,3-dione is also referred herein as “Compound 1.”

In one embodiment, the compound used in the methods provided herein is (R)-2-(2,6-dioxopiperidin-3-yl)-4-((2-fluoro-4-((3-morpholinoazetidin-1-yl)methyl)benzyl)amino)isoindoline-1,3-dione of the following formula (referred herein as “Compound 2”):

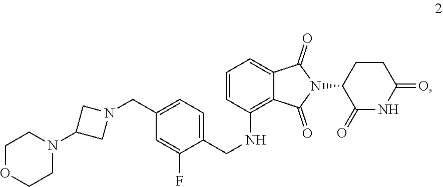 [SMILES: O=C1CC[C@@H](N2C(=O)c3cccc(NCc4ccc(CN5CC(N6CCOCC6)C5)cc4F)c3C2=O)C(=O)N1]

or tautomer, isotopolog, or pharmaceutically acceptable salt thereof.

In one embodiment, the compound used in the methods provided herein is 2-(2,6-dioxopiperidin-3-yl)-4-((2-fluoro-4-((3-morpholinoazetidin-1-yl)methyl)benzyl)amino)isoindoline-1,3-dione of the following formula (referred herein as “Compound 3”):

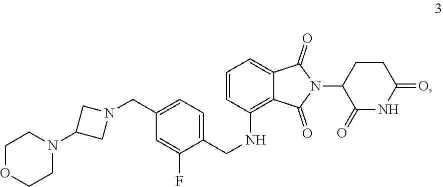 [SMILES: O=C1CCC(N2C(=O)c3cccc(NCc4ccc(CN5CC(N6CCOCC6)C5)cc4F)c3C2=O)C(=O)N1]

or tautomer, isotopolog, or pharmaceutically acceptable salt thereof.

In one embodiment, Compound 1 is used in the methods provided herein. In one embodiment, a tautomer of Compound 1 is used in the methods provided herein. In one embodiment, an isotopolog of Compound 1 is used in the methods provided herein. In one embodiment, a pharmaceutically acceptable salt of Compound 1 is used in the methods provided herein. In one embodiment, a hydrochloride salt of Compound 1 is used in the methods provided herein. In one embodiment, a mono-hydrochloride salt of Compound 1 is used in the methods provided herein. Certain salts and polymorphic forms of Compound 1 are described in U.S. patent application Ser. No. 17/075,359, the entirety of which is incorporated herein by reference.

In one embodiment, Compound 2 is used in the methods provided herein. In one embodiment, a tautomer of Compound 2 is used in the methods provided herein. In one embodiment, an isotopolog of Compound 2 is used in the methods provided herein. In one embodiment, a pharmaceutically acceptable salt of Compound 2 is used in the methods provided herein. In one embodiment, a hydrochloride salt of Compound 2 is used in the methods provided herein.

In one embodiment, Compound 3 is used in the methods provided herein. In one embodiment, an enantiomer of Compound 3 is used in the methods provided herein. In one embodiment, a mixture of enantiomers of Compound 3 is used in the methods provided herein. In one embodiment, a tautomer of Compound 3 is used in the methods provided herein. In one embodiment, an isotopolog of Compound 3 is used in the methods provided herein. In one embodiment, a pharmaceutically acceptable salt of Compound 3 is used in the methods provided herein. In one embodiment, a hydrochloride salt of Compound 3 is used in the methods provided herein.

The synthesis and certain use of the compounds provided herein are described in U.S. Patent Publication Nos. 2019/0322647 A1 and 2020/0325129 A1, and U.S. patent application Ser. Nos. 17/075,496, 17/075,523, and 17/075,125, the entirety of each of which is incorporated herein by reference.

### Second Therapeutic Agents

In one embodiment, the second therapeutic agent used in the methods provided herein is tafasitamab.

Tafasitamab is an anti-CD19 monoclonal antibody that received accelerated approval from the FDA for the treatment of R/R DLBCL in combination with lenalidomide, in transplant-ineligible patients. Approval was based on data from the L-MIND study (Salles G, et al., Lancet Oncol. 2020 July; 21(7):978-988), an open label, multicenter single arm trial that enrolled patients with R/R DLBCL who had previously received at least 1 prior systemic treatment including an anti-CD20 antibody and were not candidates for ASCT. Treatment consisted of tafasitamab 12 mg/kg intravenously in combination with lenalidomide (25 mg orally on Days 1 to 21 of each 28-day cycle) for maximum of 12 cycles, followed by tafasitamab as monotherapy until disease progression or unacceptable toxicity as follows: cycle 1, days 1, 4, 8, 15 and 22 of the 28-day cycle; cycles 2 and 3, Days 1, 8, 15 and 22 of each 28-day cycle; cycles 4 and beyond, days 1 and 15 of each 28-day cycle. Data was reported for 81 participants, with objective response rate (ORR) of 60% and complete response (CR) rate of 34%; median duration of response was 21.7 months. Serious AEs occurred in 52% of patients, including pneumonia in 6% and febrile neutropenia in 6%. The most common adverse events (AEs) of all grades (≥20%) were neutropenia, fatigue, anemia, diarrhea, thrombocytopenia, cough, pyrexia, peripheral edema, rash, and decreased appetite. G3/4 neutropenia and febrile neutropenia were reported for 48% and 12% of patients, respectively; thrombocytopenia was identified in 17% of patients and anemia in 7%. Important safety information includes warnings for IRR, myelosuppression, infections and embryo-fetal toxicity.

In one embodiment, the second therapeutic agent used in the methods provided herein is obinutuzumab.

Obinutuzumab is a CD20-directed antibody that is approved in combination with bendamustine for the treatment of follicular lymphoma patients who previously received a rituximab-containing regimen; or in combination with chemotherapy followed by obinutuzumab monotherapy in patients achieving at least a partial remission, for the treatment of adult patients with previously untreated stage II bulky, III or IV follicular lymphoma. Approval in R/R FL was based on data from the GADOLIN study (Sehn L H, et al., Lancet Onocol. 2016; 17(8)1081-1093), an open-label, randomised, phase 3 study for adult patients with CD20-positive indolent non-Hodgkin lymphoma refractory to rituximab. Patients were randomized between obinutuzumab combined with bendamustine or bendamustine monotherapy, for 6 28-day cycles consisting of obinutuzumab 1000 mg (days 1, 8, and 15, cycle 1; day 1, cycles 2-6) plus bendamustine 90 mg/m² per day (days 1 and 2, cycles 1-6), and bendamustine monotherapy 120 mg/m² per day (days 1 and 2, all cycles). Non-progressing patients in the obinutuzumab plus bendamustine group received obinutuzumab maintenance (1000 mg every 2 months) for up to 2 years. A total of 396 patients were randomized, 194 of them to obinutuzumab plus bendamustine arm. After a median follow-up of 21.9 months (obinutuzumab plus benadmustine) and 20.3 months (bendamustine monotherapy), the primary endpoint, PFS, was significantly longer with obinutuzumab plus bendamustine (NR, not reached, [95% CI 22.5 months—not estimable])) versus 14.9 months (95% CI: 12.8-16.6 months) for the bendamustine monotherapy arm (hazard ration 0.55 [95% CI: 0.40-0.74] ). Serious AEs occurred in 38% of patients in the obinutuzumab plus bendamustine group and in 33% of patients in the bendamustine monotherapy group, and deaths due to adverse events occurred in 6% of patients in each group. The most common AEs of all grades (≥20%) were nausea, fatigue, neutropenia, cough, pyrexia, diarrhea and vomiting for both arms, and in addition to that, thrombocytopenia in the monotherapy arm and constipation in the combination arm. Grade 3/4 neutropenia were reported for 26% and 33% of patients, respectively, in the monotherapy arm and combination arm; Grade 3/4 thrombocytopenia, in 15% and 17%, respectively; and anemia, in 8% and 10%, respectively. Febrile neutropenia was uncommon in both arms. Important safety information includes warnings for hepatitis B virus (HBV) reactivation and progressive multifocal leukoencephalopathy (PML) also infusion-related reactions (IRR), Tumor Lysis Syndrome (TLS). Additional safety information includes GI perforation and worsening cardiac conditions.

In one embodiment, the second therapeutic agent used in the methods provided herein is tazemetostat, or a tautomer, isotopolog, or pharmaceutically acceptable salt thereof is used in the methods provided herein. In one embodiment, the second therapeutic agent used in the methods provided herein is tazemetostat. Tazemetostat (also known as EPZ-6438) has a chemical name of N-[(1,2-dihydro-4,6-dimethyl-2-oxo-3-pyridinyl)methyl]-5-[ethyl (tetrahydro-2H-pyran-4-yl)amino]-4-methyl-4′-(4-morpholinylmethyl)-[1,1′-biphenyl]-3-carboxamide, and has the structure:

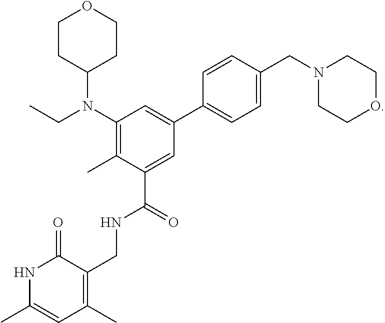 [SMILES: CCN(c1cc(-c2ccc(CN3CCOCC3)cc2)cc(C(=O)NCc2c(C)cc(C)[nH]c2=O)c1C)C1CCOCC1]

Tazemetostat is a selective inhibitor of histone methyl transferase Enhancer of Zeste Homolog 2 (EZH2), which blocks methylation of lysine 27 on histone H3, altering gene expression patterns associated with cancer pathways and resulting in decreased tumor cell proliferation (Vire E, et al., Nature. 2006; 439(7078)871-874). Somatic gain of function mutations in EZH2 have been identified in a variety of tumors, among them FL, for which studies have found up to 27% of patients with EZH2 mutated status (Bodor C, et al., Blood. 2013; 122(18)3165-3168). Tazemetostat has recently received accelerated approval from the FDA for treatment of R/R FL in adult patients whose tumors are positive for an EZH2 mutation as detected by an FDA-approved test and who have received at least 2 prior systemic therapies, and in adult patients with R/R FL who have no satisfactory alternative treatment options. Approval was based on data from 2 single-arm cohorts of a multi-center study which enrolled patients with FL who had received 2 or more prior systemic therapies. Treatment consisted of tazemetostat 800 mg orally twice daily until disease progression or discontinuation due to toxicity. ORR in 42 patients with EZH2 mutated status was 69% and CR rate was 12%; mDOR for these patients was 10.9 months. Among 53 patients with EZH2 wild-type status, ORR was 34% and CR rate was 4%; mDOR for this cohort was 13 months. Serious AEs occurred in 30% of patients, most commonly general physical health deterioration, abdominal pain, pneumonia, sepsis and anemia with ≥2% of patients. The most common AEs of all grades (≥20%) were fatigue, upper respiratory tract infection, nausea, abdominal pain and musculoskeletal pain. Grade 3/4 neutropenia was reported for 7% of patients, thrombocytopenia for 7% and anemia for 8%. Important safety information includes warnings for secondary malignancies, and embryo-fetal toxicity.

### Methods of Treatment and Prevention

In one embodiment, provided herein are methods of using (S)-2-(2,6-dioxopiperidin-3-yl)-4-((2-fluoro-4-((3-morpholinoazetidin-1-yl)methyl)benzyl)amino)isoindoline-1,3-dione, or an enantiomer, a mixture of enantiomers, a tautomer, an isotopolog, or a pharmaceutically acceptable salt thereof, in combination with a second therapeutic agent, for treating, preventing or managing NHL. In one embodiment, the second therapeutic agent is tafasitamab, obinutuzumab, or tazemetostat.

In one embodiment, provided herein is a method of treating NHL, comprising administering to a subject in need thereof a therapeutically effective amount of a compound of Formula (I):

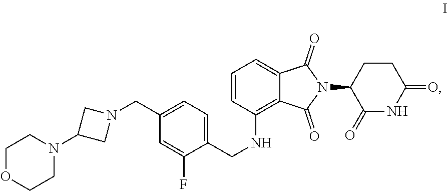 [SMILES: O=C1CC[C@H](N2C(=O)c3cccc(NCc4ccc(CN5CC(N6CCOCC6)C5)cc4F)c3C2=O)C(=O)N1]

or an enantiomer, mixture of enantiomers, tautomer, isotopolog, or pharmaceutically acceptable salt thereof (e.g., a hydrochloride salt), in combination with a second therapeutic agent, wherein the second therapeutic agent is tafasitamab, obinutuzumab, or tazemetostat. Unless otherwise specified, “a compound of Formula (I)” and “Compound 1” are used interchangeably herein.

In one embodiment, provided herein is a method of preventing NHL, which comprises administering to a subject in need thereof a therapeutically effective amount of Compound 1, or an enantiomer, mixture of enantiomers, tautomer, isotopolog, or pharmaceutically acceptable salt thereof (e.g., a hydrochloride salt), in combination with tafasitamab, obinutuzumab, or tazemetostat.

In one embodiment, provided herein is a method of managing NHL, which comprises administering to a subject in need thereof a therapeutically effective amount of Compound 1, or an enantiomer, mixture of enantiomers, tautomer, isotopolog, or pharmaceutically acceptable salt thereof (e.g., a hydrochloride salt), in combination with tafasitamab, obinutuzumab, or tazemetostat.

In certain embodiments, the NHL is diffuse large B-cell lymphoma (DLBCL). In one embodiment, the DLBCL is primary DLBCL. In one embodiment, the DLBCL is activated B-cell-like DLBCL (ABC-DLBCL). In one embodiment, the DLBCL is germinal center B-cell-like DLBCL (GCB-DLBCL). In one embodiment, the DLBCL is unclassified DLBCL. In one embodiment, the DLBCL is primary mediastinal B-cell type DLBCL (PMBL DLBCL). In one embodiment, the DLBCL is double-hit DLBCL (DHIT DLBCL), also referred to as cMyc/Bcl-2 mutant DLBCL. In one embodiment, the DLBCL is triple-hit DLBCL (THIT DLBCL) also referred to as cMyc/Bcl2/Bcl6 rearrangement DLBCL. In one embodiment, the DLBCL is DLBCL not otherwise specified (NOS) and high-grade B-cell lymphoma with MYC and BLC2 and/or BCL6 rearrangements with DLBCL morphology.

In certain embodiments, the NHL is follicular lymphoma (FL).

In other embodiments, the NHL is mantle cell lymphoma (MCL).

In yet other embodiments, the NHL is primary central nervous system lymphoma (PCNSL).

In one embodiment, the NHL is characterized by the presence of an Enhancer of Zeste Homolog 2 (EZH2) mutation. In one embodiment, the FL is characterized by the presence of EZH2 mutation. In one embodiment, the EZH2 mutation is detected by an FDA-approved test. In one embodiment, the EZH2 mutation is identified using tumor samples by whole genome sequencing (WGS). In one embodiment, the EZH2 mutation is identified using formalin-fixed, paraffin-embedded tumor samples, by whole genome sequencing (WGS).

In one embodiment, when the second therapeutic agent is tazemetostat, the NHL is follicular lymphoma (FL), primary central nervous system lymphoma (PCNSL), or mantle cell lymphoma (MCL). In one embodiment, when the second therapeutic agent is tazemetostat, the NHL is not DLBCL.

In certain embodiments, the NHL is relapsed or refractory NHL. In one embodiment, the NHL is relapsed NHL. In one embodiment, the NHL is refractory NHL.

In certain embodiments, the NHL subject has radiological evidence of progression after achieving a complete response (CR). In certain embodiments, the NHL subject has achieved less than a CR to most recent systemic therapy containing regimen, and has radiological evidence of active disease or disease progression or recurrence in less than or equal to 12 months of prior stem cell transplantation (SCT).

In certain embodiments, the NHL subject has failed one or more lines of therapy and is not a candidate for other therapy. In certain embodiments, the subject has received at least one prior therapy and is not eligible for any therapy other than the methods of treatment described herein. In certain embodiments, the subject has relapsed after or progressed on standard anticancer therapy.

In certain embodiments, the subject has received at least one prior line of therapy. In certain embodiments, the subject has received at least two prior lines of therapy. In certain embodiments, the subject has received one or more systemic regimens, and wherein one or more of the systemic regimens comprise an anti-CD20 therapy. In certain embodiments, the subject has received one to three systemic regimens, and wherein at least one of the systemic regimens is an anti-CD20 therapy.

In one embodiment, the NHL is relapsed or refractory DLBCL. In one embodiment, the DLBCL is relapsed DLBCL. In one embodiment, the DLBCL is refractory DLBCL. In one embodiment, the DLBCL is refractory to doxorubicin. In one embodiment, the DLBCL is resistant to doxorubicin.

In one embodiment, the DLBCL is treated with two or more prior lines of treatment.

In one embodiment, the DLBCL is transformed lymphoma. In another embodiment, the DLBCL is not otherwise specified (NOS) DLBCL.

In one embodiment, a subject having relapsed or refractory DLBCL has received at least one prior line of therapy. In one embodiment, a subject having relapsed or refractory DLBCL has received at least two prior lines of therapy. In one embodiment, no more than one of the prior lines of therapy may be a treatment for lower grade lymphoma. In one embodiment, a subject having relapsed or refractory DLBCL has received at least one standard treatment regimen for DLBCL. In one embodiment, a subject having relapsed or refractory DLBCL has received one or more systemic regimens, and wherein one or more of the systemic regimens comprise an anti-CD20 therapy. In certain embodiments, the subject has received one to three systemic regimens, and wherein at least one of the systemic regimens is an anti-CD20 therapy.

In one embodiment, the NHL is relapsed or refractory FL. In one embodiment, the FL is relapsed FL. In one embodiment, the FL is refractory FL.

In one embodiment, the FL is treated with one or more prior lines of treatment. In one embodiment, the FL is treated with two or more prior lines of treatment.

In one embodiment, a subject having relapsed or refractory FL has received at least one prior line of therapy. In one embodiment, a subject having relapsed or refractory FL has received at least two prior lines of therapy. In one embodiment, a subject having relapsed or refractory FL has Grade 1, 2, 3a or 3b according to Groupe d'Etude des Lymphomes Folliculaires (GELF) criteria (National Comprehensive Cancer Network. NCCN Clinical Practice Guidelines in Oncology: B-cell Lymphomas; V.2.2018. 2018 Feb. 26; V.2), the entirety of which is incorporated herein by reference. In one embodiment, a subject having relapsed or refractory FL has received one or more systemic regimens, and wherein one or more of the systemic regimens comprise an anti-CD20 therapy. In certain embodiments, the subject has received one to three systemic regimens, and wherein at least one of the systemic regimens is an anti-CD20 therapy.

In one embodiment, the NHL is relapsed or refractory MCL. In one embodiment, the MCL is relapsed MCL. In one embodiment, the MCL is refractory MCL.

In one embodiment, the MCL is treated with one or more prior lines of treatment. In one embodiment, the MCL is treated with two or more prior lines of treatment. In one embodiment, a relapsed or refractory MCL subject has progressed on or been refractory to at least one BTK inhibitor containing regimen. In one embodiment, the BTK inhibitor is ibrutinib. In one embodiment, the BTK inhibitor is acalabrutinib.

In one embodiment, the NHL is relapsed or refractory PCNSL. In one embodiment, the PCNSL is relapsed PCNSL. In one embodiment, the PCNSL is refractory PCNSL.

In one embodiment, the PCNSL is treated with one or more prior lines of treatment. In one embodiment, the PCNSL is treated with two or more prior lines of treatment.

In certain embodiments, the NHL is newly diagnosed NHL. In certain embodiments, the NHL is newly diagnosed diffuse large B-cell lymphoma. In certain embodiments, the NHL is newly diagnosed follicular lymphoma. In certain embodiments, the NHL is newly diagnosed mantle cell lymphoma. In certain embodiments, the NHL is newly diagnosed primary central nervous system lymphoma.

In one embodiment, a first therapy (e.g., a prophylactic or therapeutic agent such as Compound 1, or an enantiomer, mixture of enantiomers, tautomer, isotopolog, or pharmaceutically acceptable salt thereof) provided herein is administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before) to the administration of a second therapeutic agent (e.g., tafasitamab, obinutuzumab, or tazemetostat) to the subject.

In one embodiment, a first therapy (e.g., a prophylactic or therapeutic agent such as Compound 1, or an enantiomer, mixture of enantiomers, tautomer, isotopolog, or pharmaceutically acceptable salt thereof) provided herein is administered concomitantly with the administration of a second therapy (e.g., tafasitamab, obinutuzumab, or tazemetostat) to the subject.

In one embodiment, a first therapy (e.g., a prophylactic or therapeutic agent such as Compound 1, or an enantiomer, mixture of enantiomers, tautomer, isotopolog, or pharmaceutically acceptable salt thereof) provided herein is administered subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent (e.g., tafasitamab, obinutuzumab, or tazemetostat) to the subject.

In one embodiment, tafasitamab is administered within or up to 2 hours after the administration of a compound provided herein. In one embodiment, tafasitamab is administered concurrently with a compound provided herein. In one embodiment, obinutuzumab is administered within or up to 2 hours after the administration of a compound provided herein. In one embodiment, obinutuzumab is administered concurrently with a compound provided herein. In one embodiment, tazemetostat is administered within or up to 2 hours after the administration of a compound provided herein. In one embodiment, tazemetostat is administered concurrently with a compound provided herein.

In one embodiment, a compound described herein, e.g., Compound 1, or an enantiomer, mixture of enantiomers, tautomer, isotopolog, or pharmaceutically acceptable salt thereof (e.g., a hydrochloride salt of Compound 1), is administered at a dose of from about 0.1 mg to about 1.6 mg per day. In one embodiment, the compound is administered at a dose of from about 0.1 mg to about 1.2 mg per day. In one embodiment, the compound is administered at a dose of from about 0.1 mg to about 0.8 mg per day. In one embodiment, the compound is administered at a dose of from about 0.1 mg to about 0.6 mg per day. In one embodiment, the compound is administered at a dose of from about 0.1 mg to about 0.4 mg per day. In one embodiment, the compound is administered at a dose of from about 0.1 mg to about 0.2 mg per day. In one embodiment, the compound is administered at a dose of from about 0.2 mg to about 1.6 mg per day. In one embodiment, the compound is administered at a dose of from about 0.2 mg to about 1.2 mg per day. In one embodiment, the compound is administered at a dose of from about 0.2 mg to about 0.8 mg per day. In one embodiment, the compound is administered at a dose of from about 0.2 mg to about 0.6 mg per day. In one embodiment, the compound is administered at a dose of from about 0.2 mg to about 0.4 mg per day. In one embodiment, the compound is administered at a dose of from about 0.4 mg to about 1.6 mg per day. In one embodiment, the compound is administered at a dose of from about 0.4 mg to about 1.2 mg per day. In one embodiment, the compound is administered at a dose of from about 0.4 mg to about 0.8 mg per day. In one embodiment, the compound is administered at a dose of from about 0.4 mg to about 0.6 mg per day. In one embodiment, the compound is administered at a dose of from about 0.6 mg to about 1.6 mg per day. In one embodiment, the compound is administered at a dose of from about 0.6 mg to about 1.2 mg per day. In one embodiment, the compound is administered at a dose of from about 0.6 mg to about 0.8 mg per day. In one embodiment, the compound is administered at a dose of from about 0.8 mg to about 1.6 mg per day. In one embodiment, the compound is administered at a dose of from about 0.8 mg to about 1.2 mg per day. In one embodiment, the compound is administered at a dose of from about 1.2 mg to about 1.6 mg per day.

In certain embodiments, a compound described herein, e.g., Compound 1, or an enantiomer, mixture of enantiomers, tautomer, isotopolog, or pharmaceutically acceptable salt thereof (e.g., a hydrochloride salt of Compound 1), is administered at a dose of about 0.1 mg, about 0.2 mg, about 0.4 mg, about 0.6 mg, about 0.8 mg, about 1.2 mg, or about 1.6 mg per day. In certain embodiments, the compound is administered at a dose of about 0.1 mg per day. In certain embodiments, the compound is administered at a dose of about 0.2 mg per day. In certain embodiments, the compound is administered at a dose of about 0.4 mg per day. In certain embodiments, the compound is administered at a dose of about 0.6 mg per day. In certain embodiments, the compound is administered at a dose of about 0.8 mg per day. In certain embodiments, the compound is administered at a dose of about 1.2 mg per day. In certain embodiments, the compound is administered at a dose of about 1.6 mg per day.

In one embodiment, a compound of Formula (I), or a pharmaceutically acceptable salt thereof, is administered. In one embodiment, a hydrochloride salt of a compound of Formula (I) is administered.

In one embodiment, the second therapeutic agent is tafasitamab. In one embodiment, tafasitamab is administered in an amount according to the physician's decision. In one embodiment, tafasitamab is administered according to the locally approved label or pharmacy manual for preparation, administration, and storage information. In one embodiment, tafasitamab is administered according to the label of MONJUVI®. In one embodiment, tafasitamab is administered intravenously. In one embodiment, tafasitamab is administered via intravenous (IV) injection or IV infusion. In one embodiment, tafasitamab is administered via IV infusion.

In one embodiment, tafasitamab is administered in an amount of from about 2 mg/kg to about 25 mg/kg of the body weight of the subject. In one embodiment, tafasitamab is administered in an amount of from about 4 mg/kg to about 22 mg/kg of the body weight of the subject. In one embodiment, tafasitamab is administered in an amount of from about 6 mg/kg to about 20 mg/kg of the body weight of the subject. In one embodiment, tafasitamab is administered in an amount of from about 8 mg/kg to about 18 mg/kg of the body weight of the subject. In one embodiment, tafasitamab is administered in an amount of from about 10 mg/kg to about 16 mg/kg of the body weight of the subject. In one embodiment, tafasitamab is administered in an amount of about 12 mg/kg of the body weight of the subject.

In one embodiment, tafasitamab is administered once every 3 days, once every 4 days, once every 7 days, or once every 14 days. In one embodiment, tafasitamab is administered on days 1, 4, 8, 15, and 22 of the first 28-day cycle, days 1, 8, 15, and 22 of the second and third 28-day cycles, and days 1 and 15 of the subsequent 28-day cycle(s).

In one embodiment, the second therapeutic agent is obinutuzumab. In one embodiment, obinutuzumab is administered in an amount according to the physician's decision. In one embodiment, obinutuzumab is administered according to the locally approved label or pharmacy manual for preparation, administration, and storage information. In one embodiment, obinutuzumab is administered according to the label of GAZYVA®. In one embodiment, obinutuzumab is administered intravenously. In one embodiment, obinutuzumab is administered subcutaneously. In one embodiment, obinutuzumab is administered via intravenous (IV) injection or IV infusion. In one embodiment, obinutuzumab is administered via IV injection. In one embodiment, obinutuzumab is administered via IV infusion. In one embodiment, obinutuzumab is not administered via intravenous push or bolus.

In one embodiment, obinutuzumab is administered in an amount of from about 75 mg to about 1100 mg per day. In one embodiment, obinutuzumab is administered in an amount of from about 75 mg to about 125 mg per day, from about 80 mg to about 110 mg per day, from about 180 mg to about 210 mg per day, from about 280 mg to about 310 mg per day, from about 380 mg to about 410 mg per day, from about 480 mg to about 510 mg per day, from about 580 mg to about 610 mg per day, from about 680 mg to about 710 mg per day, from about 780 mg to about 810 mg per day, from about 800 mg to about 1200 mg per day, or from about 900 mg to about 1100 mg per day. In one embodiment, obinutuzumab is administered in an amount of from about 100 mg to about 900 mg per day. In one embodiment, obinutuzumab is administered in an amount of about 100 mg per day. In one embodiment, obinutuzumab is administered in an amount of about 900 mg per day. In one embodiment, obinutuzumab is administered in an amount of about 1000 mg per day.

In one embodiment, obinutuzumab is administered on day 1 of the first 28-day cycle, on day 2 of the first 28-day cycle, and on days 8 and 15 of the first 28-day cycle and day 1 of a second to a sixth 28-day cycles. In one embodiment, obinutuzumab is administered on days 1, 8 and 15 of the first 28-day cycle, on day 1 of the second to the sixth or eighth 28-day cycles. In one embodiment, obinutuzumab is administered on days 1, 8 and 15 of the first 28-day cycle, on day 1 of the second to the sixth or eighth 28-day cycles, and then every 2 months. In one embodiment, obinutuzumab is administered on days 1, 8 and 15 of the first 28-day cycle, on day 1 of the second to the sixth 28-day cycles or until clinically significant disease progression. In one embodiment, obinutuzumab is administered on days 1, 8 and 15 of the first 28-day cycle, on day 1 of the subsequent 28-day cycle(s).

In one embodiment, obinutuzumab is administered in an amount of about 1000 mg combining the amount administered on day 1 and day 2 (for example, about 1000 mg on day 1 and no administration on day 2, about 100 mg on day 1 and about 900 mg on day 2, and about 200 mg on day 1 and about 800 mg on day 2) of the first 28-day cycle, about 1000 mg on days 8 and 15 of the first 28-day cycle, and then about 1000 mg on day 1 of a second to a sixth 28-day cycles. In one embodiment, obinutuzumab is administered in an amount of about 1000 mg on days 1, 8 and 15 of the first 28-day cycle, about 1000 mg on day 1 of the second to the sixth or eighth 28-day cycles, and then about 1000 mg every 2 months. In one embodiment, obinutuzumab is administered in an amount of about 1000 mg on days 1, 8 and 15 of the first 28-day cycle, and about 1000 mg on day 1 of the second to the sixth 28-day cycles. In one embodiment, obinutuzumab is administered in an amount of about 1000 mg on days 1, 8 and 15 of the first 28-day cycle, about 1000 mg on day 1 of the subsequent 28-day cycles. In one embodiment, obinutuzumab is administered in an amount of about 1000 mg on days 1, 8 and 15 of the first 28-day cycle, about 1000 mg on day 1 of the second to the sixth 28-day cycles, and then about 1000 mg every 2 months.

In one embodiment, the second therapeutic agent is tazemetostat. In one embodiment, tazemetostat is administered in an amount according to the physician's decision. In one embodiment, tazemetostat is administered according to the locally approved label or pharmacy manual for preparation, administration, and storage information. In one embodiment, tazemetostat is administered according to the label of TAZVERIK. In one embodiment, tazemetostat is administered orally. In one embodiment, tazemetostat is administered with or without food. In one embodiment, tazemetostat is administered when the subject is fed. In one embodiment, tazemetostat is administered when the subject is fasted.

In one embodiment, tazemetostat is administered in an amount of from about 150 mg to about 250 mg per day. In one embodiment, tazemetostat is administered in an amount of about 200 mg per day. In one embodiment, tazemetostat is administered in an amount of from about 350 mg to about 450 mg per day. In one embodiment, tazemetostat is administered in an amount of about 400 mg per day. In one embodiment, tazemetostat is administered in an amount of from about 550 mg to about 650 mg per day. In one embodiment, tazemetostat is administered in an amount of about 600 mg per day. In one embodiment, tazemetostat is administered in an amount of from about 700 mg to about 900 mg per day. In one embodiment, tazemetostat is administered in an amount of about 800 mg per day. In one embodiment, tazemetostat is administered in an amount of from about 1000 mg to about 1400 mg per day. In one embodiment, tazemetostat is administered in an amount of about 1200 mg per day. In one embodiment, tazemetostat is administered in an amount of from about 1400 mg to about 1800 mg per day. In one embodiment, tazemetostat is administered in an amount of about 1600 mg per day. In one embodiment, tazemetostat is not co-administered with a strong or moderate CYP3A inhibitor.

In one embodiment, tazemetostat is administered once daily. In one embodiment, tazemetostat is administered twice daily. In one embodiment, tazemetostat is administered three times a day. In one embodiment, tazemetostat is administered in an amount of about 400 mg twice daily. In one embodiment, tazemetostat is administered in an amount of about 600 mg twice daily. In one embodiment, tazemetostat is administered in an amount of about 800 mg twice daily.

In one embodiment, after administration of the combination of Compound 1, or a pharmaceutically acceptable salt thereof (e.g., a hydrochloride salt of Compound 1) and a second therapeutic agent provided herein (e.g., obinutuzumab) in one or more cycles as described herein (combination therapy cycles), the method provided herein further comprises administering Compound 1, or a pharmaceutically acceptable salt thereof (e.g., a hydrochloride salt of Compound 1) in one or more cycles (monotherapy cycles). In one embodiment, the monotherapy cycles comprises one or more 28-day cycles. In one embodiment, the compound is administered for 5 days followed by 2 days of rest, starting on day 1 of the 28-day cycle. In one embodiment, the compound is administered for 5 days followed by 9 days of rest, starting on day 1 of the 28-day cycle. In one embodiment, the compound is administered for 7 days followed by 7 days of rest, starting on day 1 of the 28-day cycle. In one embodiment, the compound is administered for 10 days followed by 4 days of rest, starting on day 1 of the 28-day cycle. In one embodiment, the compound is administered for 14 days followed by 14 days of rest, starting on day 1 of the 28-day cycle. In one embodiment, the compound is administered for 21 days followed by 7 days of rest, starting on day 1 of the 28-day cycle.

In one embodiment, the method further comprises administering to the subject a growth factor. In one embodiment, the growth factor is administered for prophylactic purpose (e.g., to prevent a subject from developing neutropenia (e.g., grade 3/4 neutropenia, prolonged severe neutropenia, febrile neutropenia)). In one embodiment, the growth factor is administered for therapeutic purpose (e.g., to treat or manage neutropenia (e.g., grade 3/4 neutropenia, prolonged severe neutropenia, febrile neutropenia) in a subject that developed neutropenia). In one embodiment, the method further comprises administering to the subject granulocyte-colony stimulating factor (G-CSF) or granulocyte-macrophage colony stimulating factor (GM-CSF).

In one embodiment, G-CSF is administered in an amount according to the physician's decision. In one embodiment, G-CSF is administered according to the locally approved label or pharmacy manual for preparation, administration, and storage information. In one embodiment, G-CSF is administered according to the label of NEUPOGEN® (filgrastim). In one embodiment, G-CSF is administered according to the label of NEULASTA® (pegfilgrastim). In one embodiment, G-CSF is administered subcutaneously. In one embodiment, G-CSF is administered via subcutaneous injection. In one embodiment, G-CSF is administered intravenously. In one embodiment, G-CSF is administered via intravenous (IV) injection. In one embodiment, G-CSF is administered via IV infusion.

In one embodiment, G-CSF is filgrastim. In one embodiment, filgrastim is administered in an amount of from about 8 mcg/day/kg to about 12 mcg/day/kg of the body weight of the subject. In one embodiment, filgrastim is administered in an amount of about 10 mcg/day/kg of the body weight of the subject. In one embodiment, filgrastim is administered in an amount of from about 4 mcg/day/kg to about 8 mcg/day/kg of the body weight of the subject. In one embodiment, filgrastim is administered in an amount of about 6 mcg/day/kg of the body weight of the subject. In one embodiment, filgrastim is administered in an amount of about 5 mcg/day/kg of the body weight of the subject.

In one embodiment, G-CSF is pegfilgrastim. In one embodiment, pegfilgrastim is administered in an amount of from about 4 mg to about 8 mg. In one embodiment, pegfilgrastim is administered in an amount of about 6 mg. In one embodiment, pegfilgrastim is administered in an amount of about 4 mg.

In one embodiment, GM-CSF is administered in an amount according to the physician's decision. In one embodiment, GM-CSF is administered according to the locally approved label or pharmacy manual for preparation, administration, and storage information. In one embodiment, GM-CSF is administered according to the label of LEUKINE® (sargramostim). In one embodiment, GM-CSF is administered subcutaneously. In one embodiment, GM-CSF is administered via subcutaneous injection. In one embodiment, GM-CSF is administered intravenously. In one embodiment, GM-CSF is administered via intravenous (IV) injection. In one embodiment, GM-CSF is administered via IV infusion. In one embodiment, GM-CSF is administered intravenously over a 2-hour, 4-hour, 6-hour, 8-hour, 10-hour, or 24-hour period of time.

In one embodiment, GM-CSF is administered in an amount of about 200 mcg/m²/day to about 300 mcg/m²/day. In one embodiment, GM-CSF is administered in an amount of about 220 mcg/m²/day to about 280 mcg/m²/day. In one embodiment, GM-CSF is administered in an amount of about 250 mcg/m²/day. In one embodiment, GM-CSF is administered in an amount of from about 5 mcg/kg to about 15 mcg/kg of the body weight of the subject. In one embodiment, GM-CSF is administered in an amount of from about 7 mcg/kg of the body weight of the subject. In one embodiment, GM-CSF is administered in an amount of from about 10 mcg/kg of the body weight of the subject. In one embodiment, GM-CSF is administered in an amount of from about 12 mcg/kg of the body weight of the subject.

In one embodiment, G-CSF or GM-CSF is administered once or twice daily. In one embodiment, G-CSF or GM-CSF is administered twice a week. In one embodiment, G-CSF or GM-CSF is administered once a week.

In one embodiment, G-CSF or GM-CSF is administered during non-dosing period of a compound provided herein. In one embodiment, G-CSF or GM-CSF is administered during non-dosing period of a compound provided herein in the first 28-day cycle of dosing. In one embodiment, the G-CSF is administered on days 6 to 7 of a 7-day cycle. In one embodiment, the G-CSF or GM-CSF is administered on days 6 to 14 of a 14-day cycle. In one embodiment, the G-CSF or GM-CSF is administered on days 8 to 14 of a 14-day cycle. In one embodiment, the G-CSF or GM-CSF is administered on days 11 to 14 of a 14-day cycle. In one embodiment, the G-CSF or GM-CSF is administered on days 15 to 28 of a 28-day cycle. In one embodiment, the G-CSF or GM-CSF is administered on days 8 to 14 and days 22 to 28 of a 28-day cycle. In one embodiment, the G-CSF or GM-CSF is administered twice a week during days 8 to 14 and days 22 to 28 of a 28-day cycle. In one embodiment, the G-CSF or GM-CSF is administered twice a week during days 15 to 28 of a 28-day cycle.

In one embodiment, G-CSF or GM-CSF is administered during non-dosing period of a compound provided herein. In one embodiment, the G-CSF is administered on days 6 to 7 of a 7-day cycle whereas the compound is administered on days 1 to 5 of the 7-day cycle. In one embodiment, the G-CSF or GM-CSF is administered on days 6 to 14 of a 14-day cycle whereas the compound is administered on days 1 to 5 of the 14-day cycle. In one embodiment, the G-CSF or GM-CSF is administered on days 8 to 14 of a 14-day cycle whereas the compound is administered on days 1 to 7 of the 14-day cycle. In one embodiment, the G-CSF or GM-CSF is administered on days 11 to 14 of a 14-day cycle whereas the compound is administered on days 1 to 10 of the 14-day cycle. In one embodiment, the G-CSF or GM-CSF is administered on days 15 to 28 of a 28-day cycle whereas the compound is administered on days 1 to 14 of the 28-day cycle. In one embodiment, the G-CSF or GM-CSF is administered on days 8 to 14 and days 22 to 28 of a 28-day cycle whereas the compound is administered on days 1 to 7 and days 15 to 21 of the 28-day cycle.

In one embodiment, provided herein is a method of treating DLBCL, which comprises administering to a subject in need thereof a therapeutically effective amount of Compound 1, or an enantiomer, mixture of enantiomers, tautomer, isotopolog, or pharmaceutically acceptable salt thereof, in combination with tafasitamab, obinutuzumab, or tazemetostat. In one embodiment, provided herein is a method of treating DLBCL comprising administering Compound 1 or pharmaceutically acceptable salt thereof (e.g., a hydrochloride salt of Compound 1) in combination with tafasitamab. In one embodiment, provided herein is a method of treating DLBCL comprising administering Compound 1 or pharmaceutically acceptable salt thereof (e.g., a hydrochloride salt of Compound 1) in combination with obinutuzumab. In one embodiment, provided herein is a method of treating DLBCL comprising administering Compound 1 or pharmaceutically acceptable salt thereof (e.g., a hydrochloride salt of Compound 1) in combination with tazemetostat. In one embodiment, the method further comprises administering to the subject a growth factor.

In one embodiment, provided herein is a method of preventing DLBCL, which comprises administering to a subject in need thereof a therapeutically effective amount of a compound provided herein, e.g., Compound 1, or an enantiomer, mixture of enantiomers, tautomer, isotopolog, or pharmaceutically acceptable salt (e.g., a hydrochloride salt) thereof, in combination with tafasitamab, obinutuzumab, or tazemetostat. In one embodiment, provided herein is a method of preventing DLBCL comprising administering Compound 1, or a pharmaceutically acceptable salt thereof (e.g., a hydrochloride salt of Compound 1) in combination with tafasitamab. In one embodiment, provided herein is a method of preventing DLBCL comprising administering Compound 1, or a pharmaceutically acceptable salt thereof (e.g., a hydrochloride salt of Compound 1) in combination with obinutuzumab. In one embodiment, provided herein is a method of preventing DLBCL comprising administering Compound 1, or a pharmaceutically acceptable salt thereof (e.g., a hydrochloride salt of Compound 1) in combination with tazemetostat. In one embodiment, the method further comprises administering to the subject a growth factor.

In another embodiment, provided herein is a method of managing DLBCL, which comprises administering to a subject in need thereof a therapeutically effective amount of a compound provided herein, e.g., Compound 1, or an enantiomer, mixture of enantiomers, tautomer, isotopolog, or a pharmaceutically acceptable salt (e.g., a hydrochloride salt) thereof, in combination with tafasitamab, obinutuzumab, or tazemetostat. In one embodiment, provided herein is a method of managing DLBCL comprising administering Compound 1, or a pharmaceutically acceptable salt thereof (e.g., a hydrochloride salt of Compound 1) in combination with tafasitamab. In one embodiment, provided herein is a method of managing DLBCL comprising administering Compound 1, or a pharmaceutically acceptable salt thereof (e.g., a hydrochloride salt of Compound 1) in combination with obinutuzumab. In one embodiment, provided herein is a method of managing DLBCL comprising administering Compound 1, or a pharmaceutically acceptable salt thereof (e.g., a hydrochloride salt of Compound 1) in combination with tazemetostat. In one embodiment, the method further comprises administering to the subject a growth factor.

In one embodiment, provided herein is a method of treating FL, which comprises administering to a subject in need thereof a therapeutically effective amount of Compound 1, or an enantiomer, mixture of enantiomers, tautomer, isotopolog, or a pharmaceutically acceptable salt thereof, in combination with tafasitamab, obinutuzumab, or tazemetostat. In one embodiment, provided herein is a method of treating FL comprising administering Compound 1 or a pharmaceutically acceptable salt thereof (e.g., a hydrochloride salt of Compound 1) in combination with tafasitamab. In one embodiment, provided herein is a method of treating FL comprising administering Compound 1 or a pharmaceutically acceptable salt thereof (e.g., a hydrochloride salt of Compound 1) in combination with obinutuzumab. In one embodiment, provided herein is a method of treating FL comprising administering Compound 1 or a pharmaceutically acceptable salt thereof (e.g., a hydrochloride salt of Compound 1) in combination with tazemetostat. In one embodiment, the method further comprises administering to the subject a growth factor.

In one embodiment, provided herein is a method of preventing FL, which comprises administering to a subject in need thereof a therapeutically effective amount of a compound provided herein, e.g., Compound 1, or an enantiomer, mixture of enantiomers, tautomer, isotopolog, or a pharmaceutically acceptable salt (e.g., a hydrochloride salt) thereof, in combination with tafasitamab, obinutuzumab, or tazemetostat. In one embodiment, provided herein is a method of preventing FL comprising administering Compound 1, or a pharmaceutically acceptable salt thereof (e.g., a hydrochloride salt of Compound 1) in combination with tafasitamab. In one embodiment, provided herein is a method of preventing FL comprising administering Compound 1, or a pharmaceutically acceptable salt thereof (e.g., a hydrochloride salt of Compound 1) in combination with obinutuzumab. In one embodiment, provided herein is a method of preventing FL comprising administering Compound 1, or a pharmaceutically acceptable salt thereof (e.g., a hydrochloride salt of Compound 1) in combination with tazemetostat. In one embodiment, the method further comprises administering to the subject a growth factor.

In another embodiment, provided herein is a method of managing FL, which comprises administering to a subject in need thereof a therapeutically effective amount of a compound provided herein, e.g., Compound 1, or an enantiomer, mixture of enantiomers, tautomer, isotopolog, or a pharmaceutically acceptable salt (e.g., a hydrochloride salt) thereof, in combination with tafasitamab, obinutuzumab, or tazemetostat. In one embodiment, provided herein is a method of managing FL comprising administering Compound 1, or a pharmaceutically acceptable salt thereof (e.g., a hydrochloride salt of Compound 1) in combination with tafasitamab. In one embodiment, provided herein is a method of managing FL comprising administering Compound 1, or a pharmaceutically acceptable salt thereof (e.g., a hydrochloride salt of Compound 1) in combination with obinutuzumab. In one embodiment, provided herein is a method of managing FL comprising administering Compound 1, or a pharmaceutically acceptable salt thereof (e.g., a hydrochloride salt of Compound 1) in combination with tazemetostat. In one embodiment, the method further comprises administering to the subject a growth factor.

In one embodiment, provided herein is a method of treating MCL, which comprises administering to a subject in need thereof a therapeutically effective amount of Compound 1, or an enantiomer, mixture of enantiomers, tautomer, isotopolog, or a pharmaceutically acceptable salt thereof, in combination with tafasitamab, obinutuzumab, or tazemetostat. In one embodiment, provided herein is a method of treating MCL comprising administering Compound 1 or a pharmaceutically acceptable salt thereof (e.g., a hydrochloride salt of Compound 1) in combination with tafasitamab. In one embodiment, provided herein is a method of treating MCL comprising administering Compound 1 or a pharmaceutically acceptable salt thereof (e.g., a hydrochloride salt of Compound 1) in combination with obinutuzumab. In one embodiment, provided herein is a method of treating MCL comprising administering Compound 1 or a pharmaceutically acceptable salt thereof (e.g., a hydrochloride salt of Compound 1) in combination with tazemetostat. In one embodiment, the method further comprises administering to the subject a growth factor.

In one embodiment, provided herein is a method of preventing MCL, which comprises administering to a subject in need thereof a therapeutically effective amount of a compound provided herein, e.g., Compound 1, or an enantiomer, mixture of enantiomers, tautomer, isotopolog, or a pharmaceutically acceptable salt (e.g., a hydrochloride salt) thereof, in combination with tafasitamab, obinutuzumab, or tazemetostat. In one embodiment, provided herein is a method of preventing MCL comprising administering Compound 1, pharmaceutically acceptable salt thereof (e.g., a hydrochloride salt of Compound 1) in combination with tafasitamab. In one embodiment, provided herein is a method of preventing MCL comprising administering Compound 1, or a pharmaceutically acceptable salt thereof (e.g., a hydrochloride salt of Compound 1) in combination with obinutuzumab. In one embodiment, provided herein is a method of preventing MCL comprising administering Compound 1, or a pharmaceutically acceptable salt thereof (e.g., a hydrochloride salt of Compound 1) in combination with tazemetostat. In one embodiment, the method further comprises administering to the subject a growth factor.

In another embodiment, provided herein is a method of managing MCL, which comprises administering to a subject in need thereof a therapeutically effective amount of a compound provided herein, e.g., Compound 1, or an enantiomer, mixture of enantiomers, tautomer, isotopolog, or a pharmaceutically acceptable salt (e.g., a hydrochloride salt) thereof, in combination with tafasitamab, obinutuzumab, or tazemetostat. In one embodiment, provided herein is a method of managing MCL comprising administering Compound 1, or a pharmaceutically acceptable salt thereof (e.g., a hydrochloride salt of Compound 1) in combination with tafasitamab. In one embodiment, provided herein is a method of managing MCL comprising administering Compound 1, or a pharmaceutically acceptable salt thereof (e.g., a hydrochloride salt of Compound 1) in combination with obinutuzumab. In one embodiment, provided herein is a method of managing MCL comprising administering Compound 1, or a pharmaceutically acceptable salt thereof (e.g., a hydrochloride salt of Compound 1) in combination with tazemetostat. In one embodiment, the method further comprises administering to the subject a growth factor.

In one embodiment, provided herein is a method of treating PCNSL, which comprises administering to a subject in need thereof a therapeutically effective amount of Compound 1, or an enantiomer, mixture of enantiomers, tautomer, isotopolog, or a pharmaceutically acceptable salt thereof, in combination with tafasitamab, obinutuzumab, or tazemetostat. In one embodiment, provided herein is a method of treating PCNSL comprising administering Compound 1 or a pharmaceutically acceptable salt thereof (e.g., a hydrochloride salt of Compound 1) in combination with tafasitamab. In one embodiment, provided herein is a method of treating PCNSL comprising administering Compound 1 or a pharmaceutically acceptable salt thereof (e.g., a hydrochloride salt of Compound 1) in combination with obinutuzumab. In one embodiment, provided herein is a method of treating PCNSL comprising administering Compound 1 or a pharmaceutically acceptable salt thereof (e.g., a hydrochloride salt of Compound 1) in combination with tazemetostat. In one embodiment, the method further comprises administering to the subject a growth factor.

In one embodiment, provided herein is a method of preventing PCNSL, which comprises administering to a subject in need thereof a therapeutically effective amount of a compound provided herein, e.g., Compound 1, or an enantiomer, mixture of enantiomers, tautomer, isotopolog, or a pharmaceutically acceptable salt (e.g., a hydrochloride salt) thereof, in combination with tafasitamab, obinutuzumab, or tazemetostat. In one embodiment, provided herein is a method of preventing PCNSL comprising administering Compound 1, pharmaceutically acceptable salt thereof (e.g., a hydrochloride salt of Compound 1) in combination with tafasitamab. In one embodiment, provided herein is a method of preventing PCNSL comprising administering Compound 1, or a pharmaceutically acceptable salt thereof (e.g., a hydrochloride salt of Compound 1) in combination with obinutuzumab. In one embodiment, provided herein is a method of preventing PCNSL comprising administering Compound 1, or a pharmaceutically acceptable salt thereof (e.g., a hydrochloride salt of Compound 1) in combination with tazemetostat. In one embodiment, the method further comprises administering to the subject a growth factor.

In another embodiment, provided herein is a method of managing PCNSL, which comprises administering to a subject in need thereof a therapeutically effective amount of a compound provided herein, e.g., Compound 1, or an enantiomer, mixture of enantiomers, tautomer, isotopolog, or pharmaceutically acceptable salt (e.g., a hydrochloride salt) thereof, in combination with tafasitamab, obinutuzumab, or tazemetostat. In one embodiment, provided herein is a method of managing PCNSL comprising administering Compound 1, or a pharmaceutically acceptable salt thereof (e.g., a hydrochloride salt of Compound 1) in combination with tafasitamab. In one embodiment, provided herein is a method of managing PCNSL comprising administering Compound 1, or a pharmaceutically acceptable salt thereof (e.g., a hydrochloride salt of Compound 1) in combination with obinutuzumab. In one embodiment, provided herein is a method of managing PCNSL comprising administering Compound 1, or a pharmaceutically acceptable salt thereof (e.g., a hydrochloride salt of Compound 1) in combination with tazemetostat. In one embodiment, the method further comprises administering to the subject a growth factor.

In one embodiment, provided herein is a method of treating relapsed or refractory DLBCL, which comprises administering to a subject in need thereof a therapeutically effective amount of Compound 1, or an enantiomer, mixture of enantiomers, tautomer, isotopolog, or pharmaceutically acceptable salt thereof, in combination with tafasitamab, obinutuzumab, or tazemetostat. In one embodiment, provided herein is a method of treating relapsed or refractory DLBCL comprising administering Compound 1 or a pharmaceutically acceptable salt thereof (e.g., a hydrochloride salt of Compound 1) in combination with tafasitamab. In one embodiment, provided herein is a method of treating relapsed or refractory DLBCL comprising administering Compound 1 or a pharmaceutically acceptable salt thereof (e.g., a hydrochloride salt of Compound 1) in combination with obinutuzumab. In one embodiment, provided herein is a method of treating relapsed or refractory DLBCL comprising administering Compound 1 or a pharmaceutically acceptable salt thereof (e.g., a hydrochloride salt of Compound 1) in combination with tazemetostat. In one embodiment, the method further comprises administering to the subject a growth factor.

In one embodiment, provided herein is a method of preventing relapsed or refractory DLBCL, which comprises administering to a subject in need thereof a therapeutically effective amount of a compound provided herein, e.g., Compound 1, or an enantiomer, mixture of enantiomers, tautomer, isotopolog, or pharmaceutically acceptable salt (e.g., a hydrochloride salt) thereof, in combination with tafasitamab, obinutuzumab, or tazemetostat. In one embodiment, provided herein is a method of preventing relapsed or refractory DLBCL comprising administering Compound 1, pharmaceutically acceptable salt thereof (e.g., a hydrochloride salt of Compound 1) in combination with tafasitamab. In one embodiment, provided herein is a method of preventing relapsed or refractory DLBCL comprising administering Compound 1, or a pharmaceutically acceptable salt thereof (e.g., a hydrochloride salt of Compound 1) in combination with obinutuzumab. In one embodiment, provided herein is a method of preventing relapsed or refractory DLBCL comprising administering Compound 1, or a pharmaceutically acceptable salt thereof (e.g., a hydrochloride salt of Compound 1) in combination with tazemetostat. In one embodiment, the method further comprises administering to the subject a growth factor.

In another embodiment, provided herein is a method of managing relapsed or refractory DLBCL, which comprises administering to a subject in need thereof a therapeutically effective amount of a compound provided herein, e.g., Compound 1, or an enantiomer, mixture of enantiomers, tautomer, isotopolog, or pharmaceutically acceptable salt (e.g., a hydrochloride salt) thereof, in combination with tafasitamab, obinutuzumab, or tazemetostat. In one embodiment, provided herein is a method of managing relapsed or refractory DLBCL comprising administering Compound 1, or a pharmaceutically acceptable salt thereof (e.g., a hydrochloride salt of Compound 1) in combination with tafasitamab. In one embodiment, provided herein is a method of managing relapsed or refractory DLBCL comprising administering Compound 1, or a pharmaceutically acceptable salt thereof (e.g., a hydrochloride salt of Compound 1) in combination with obinutuzumab. In one embodiment, provided herein is a method of managing relapsed or refractory DLBCL comprising administering Compound 1, or a pharmaceutically acceptable salt thereof (e.g., a hydrochloride salt of Compound 1) in combination with tazemetostat. In one embodiment, the method further comprises administering to the subject a growth factor.

In one embodiment, provided herein is a method of treating relapsed or refractory FL, which comprises administering to a subject in need thereof a therapeutically effective amount of Compound 1, or an enantiomer, mixture of enantiomers, tautomer, isotopolog, or pharmaceutically acceptable salt thereof, in combination with tafasitamab, obinutuzumab, or tazemetostat. In one embodiment, provided herein is a method of treating relapsed or refractory FL comprising administering Compound 1 or a pharmaceutically acceptable salt thereof (e.g., a hydrochloride salt of Compound 1) in combination with tafasitamab. In one embodiment, provided herein is a method of treating relapsed or refractory FL comprising administering Compound 1 or a pharmaceutically acceptable salt thereof (e.g., a hydrochloride salt of Compound 1) in combination with obinutuzumab. In one embodiment, provided herein is a method of treating relapsed or refractory FL comprising administering Compound 1 or a pharmaceutically acceptable salt thereof (e.g., a hydrochloride salt of Compound 1) in combination with tazemetostat. In one embodiment, the method further comprises administering to the subject a growth factor.

In one embodiment, provided herein is a method of preventing relapsed or refractory FL, which comprises administering to a subject in need thereof a therapeutically effective amount of a compound provided herein, e.g., Compound 1, or an enantiomer, mixture of enantiomers, tautomer, isotopolog, or pharmaceutically acceptable salt (e.g., a hydrochloride salt) thereof, in combination with tafasitamab, obinutuzumab, or tazemetostat. In one embodiment, provided herein is a method of preventing relapsed or refractory FL comprising administering Compound 1, or a pharmaceutically acceptable salt thereof (e.g., a hydrochloride salt of Compound 1) in combination with tafasitamab. In one embodiment, provided herein is a method of preventing relapsed or refractory FL comprising administering Compound 1, or a pharmaceutically acceptable salt thereof (e.g., a hydrochloride salt of Compound 1) in combination with obinutuzumab. In one embodiment, provided herein is a method of preventing relapsed or refractory FL comprising administering Compound 1, Compound 2, Compound 3 or a pharmaceutically acceptable salt thereof (e.g., a hydrochloride salt of Compound 1) in combination with tazemetostat. In one embodiment, the method further comprises administering to the subject a growth factor.

In another embodiment, provided herein is a method of managing relapsed or refractory FL, which comprises administering to a subject in need thereof a therapeutically effective amount of a compound provided herein, e.g., Compound 1, or an enantiomer, mixture of enantiomers, tautomer, isotopolog, or pharmaceutically acceptable salt (e.g., a hydrochloride salt) thereof, in combination with tafasitamab, obinutuzumab, or tazemetostat. In one embodiment, provided herein is a method of managing relapsed or refractory FL comprising administering Compound 1, or a pharmaceutically acceptable salt thereof (e.g., a hydrochloride salt of Compound 1) in combination with tafasitamab. In one embodiment, provided herein is a method of managing relapsed or refractory FL comprising administering Compound 1, or a pharmaceutically acceptable salt thereof (e.g., a hydrochloride salt of Compound 1) in combination with obinutuzumab. In one embodiment, provided herein is a method of managing relapsed or refractory FL comprising administering Compound 1, or a pharmaceutically acceptable salt thereof (e.g., a hydrochloride salt of Compound 1) in combination with tazemetostat. In one embodiment, the method further comprises administering to the subject a growth factor.

In one embodiment, provided herein is a method of treating relapsed or refractory MCL, which comprises administering to a subject in need thereof a therapeutically effective amount of Compound 1, or an enantiomer, mixture of enantiomers, tautomer, isotopolog, or pharmaceutically acceptable salt thereof, in combination with tafasitamab, obinutuzumab, or tazemetostat. In one embodiment, provided herein is a method of treating relapsed or refractory MCL comprising administering Compound 1 or a pharmaceutically acceptable salt thereof (e.g., a hydrochloride salt of Compound 1) in combination with tafasitamab. In one embodiment, provided herein is a method of treating relapsed or refractory MCL comprising administering Compound 1 or a pharmaceutically acceptable salt thereof (e.g., a hydrochloride salt of Compound 1) in combination with obinutuzumab. In one embodiment, provided herein is a method of treating relapsed or refractory MCL comprising administering Compound 1 or a pharmaceutically acceptable salt thereof (e.g., a hydrochloride salt of Compound 1) in combination with tazemetostat. In one embodiment, the method further comprises administering to the subject a growth factor.

In one embodiment, provided herein is a method of preventing relapsed or refractory MCL, which comprises administering to a subject in need thereof a therapeutically effective amount of a compound provided herein, e.g., Compound 1, or an enantiomer, mixture of enantiomers, tautomer, isotopolog, or pharmaceutically acceptable salt (e.g., a hydrochloride salt) thereof, in combination with tafasitamab, obinutuzumab, or tazemetostat. In one embodiment, provided herein is a method of preventing relapsed or refractory MCL comprising administering Compound 1, pharmaceutically acceptable salt thereof (e.g., a hydrochloride salt of Compound 1) in combination with tafasitamab. In one embodiment, provided herein is a method of preventing relapsed or refractory MCL comprising administering Compound 1, or a pharmaceutically acceptable salt thereof (e.g., a hydrochloride salt of Compound 1) in combination with obinutuzumab. In one embodiment, provided herein is a method of preventing relapsed or refractory MCL comprising administering Compound 1, or a pharmaceutically acceptable salt thereof (e.g., a hydrochloride salt of Compound 1) in combination with tazemetostat. In one embodiment, the method further comprises administering to the subject a growth factor.

In another embodiment, provided herein is a method of managing relapsed or refractory MCL, which comprises administering to a subject in need thereof a therapeutically effective amount of a compound provided herein, e.g., Compound 1, or an enantiomer, mixture of enantiomers, tautomer, isotopolog, or pharmaceutically acceptable salt (e.g., a hydrochloride salt) thereof, in combination with tafasitamab, obinutuzumab, or tazemetostat. In one embodiment, provided herein is a method of managing relapsed or refractory MCL comprising administering Compound 1, or a pharmaceutically acceptable salt thereof (e.g., a hydrochloride salt of Compound 1) in combination with tafasitamab. In one embodiment, provided herein is a method of managing relapsed or refractory MCL comprising administering Compound 1, or a pharmaceutically acceptable salt thereof (e.g., a hydrochloride salt of Compound 1) in combination with obinutuzumab. In one embodiment, provided herein is a method of managing relapsed or refractory MCL comprising administering Compound 1, or a pharmaceutically acceptable salt thereof (e.g., a hydrochloride salt of Compound 1) in combination with tazemetostat. In one embodiment, the method further comprises administering to the subject a growth factor.

In one embodiment, provided herein is a method of treating relapsed or refractory PCNSL, which comprises administering to a subject in need thereof a therapeutically effective amount of Compound 1, or an enantiomer, mixture of enantiomers, tautomer, isotopolog, or pharmaceutically acceptable salt thereof, in combination with tafasitamab, obinutuzumab, or tazemetostat. In one embodiment, provided herein is a method of treating relapsed or refractory PCNSL comprising administering Compound 1 or a pharmaceutically acceptable salt thereof (e.g., a hydrochloride salt of Compound 1) in combination with tafasitamab. In one embodiment, provided herein is a method of treating relapsed or refractory PCNSL comprising administering Compound 1 or a pharmaceutically acceptable salt thereof (e.g., a hydrochloride salt of Compound 1) in combination with obinutuzumab. In one embodiment, provided herein is a method of treating relapsed or refractory PCNSL comprising administering Compound 1 or a pharmaceutically acceptable salt thereof (e.g., a hydrochloride salt of Compound 1) in combination with tazemetostat. In one embodiment, the method further comprises administering to the subject a growth factor.

In one embodiment, provided herein is a method of preventing relapsed or refractory PCNSL, which comprises administering to a subject in need thereof a therapeutically effective amount of a compound provided herein, e.g., Compound 1, or an enantiomer, mixture of enantiomers, tautomer, isotopolog, or pharmaceutically acceptable salt (e.g., a hydrochloride salt) thereof, in combination with tafasitamab, obinutuzumab, or tazemetostat. In one embodiment, provided herein is a method of preventing relapsed or refractory PCNSL comprising administering Compound 1, pharmaceutically acceptable salt thereof (e.g., a hydrochloride salt of Compound 1) in combination with tafasitamab. In one embodiment, provided herein is a method of preventing relapsed or refractory PCNSL comprising administering Compound 1, or a pharmaceutically acceptable salt thereof (e.g., a hydrochloride salt of Compound 1) in combination with obinutuzumab. In one embodiment, provided herein is a method of preventing relapsed or refractory PCNSL comprising administering Compound 1, or a pharmaceutically acceptable salt thereof (e.g., a hydrochloride salt of Compound 1) in combination with tazemetostat. In one embodiment, the method further comprises administering to the subject a growth factor.

In another embodiment, provided herein is a method of managing relapsed or refractory PCNSL, which comprises administering to a subject in need thereof a therapeutically effective amount of a compound provided herein, e.g., Compound 1, or an enantiomer, mixture of enantiomers, tautomer, isotopolog, or pharmaceutically acceptable salt (e.g., a hydrochloride salt) thereof, in combination with tafasitamab, obinutuzumab, or tazemetostat. In one embodiment, provided herein is a method of managing relapsed or refractory PCNSL comprising administering Compound 1, or a pharmaceutically acceptable salt thereof (e.g., a hydrochloride salt of Compound 1) in combination with tafasitamab. In one embodiment, provided herein is a method of managing relapsed or refractory PCNSL comprising administering Compound 1, or a pharmaceutically acceptable salt thereof (e.g., a hydrochloride salt of Compound 1) in combination with obinutuzumab. In one embodiment, provided herein is a method of managing relapsed or refractory PCNSL comprising administering Compound 1, or a pharmaceutically acceptable salt thereof (e.g., a hydrochloride salt of Compound 1) in combination with tazemetostat. In one embodiment, the method further comprises administering to the subject a growth factor.

In another embodiment, provided herein are methods for achieving a complete response, partial response, or stable disease, as determined by the Lugano response criteria in a patient, comprising administering an effective amount of a compound described herein, e.g., Compound 1, or an enantiomer, mixture of enantiomers, tautomer, isotopolog, or pharmaceutically acceptable salt thereof (e.g., a hydrochloride salt of Compound 1), in combination with tafasitamab, obinutuzumab, or tazemetostat, to patient having NHL. In another embodiment, provided herein are methods for achieving an increase in overall survival, progression-free survival, event-free survival, time to progression, or disease-free survival in a patient, comprising administering an effective amount of a compound described herein, e.g., Compound 1, or an enantiomer, mixture of enantiomers, tautomer, isotopolog, or pharmaceutically acceptable salt thereof (e.g., a hydrochloride salt of Compound 1), in combination with tafasitamab, obinutuzumab, or tazemetostat, to patient having NHL. In another embodiment, provided herein are methods for achieving an increase in overall survival in a patient, comprising administering an effective amount of a compound described herein, e.g., Compound 1, or an enantiomer, mixture of enantiomers, tautomer, isotopolog, or pharmaceutically acceptable salt thereof (e.g., a hydrochloride salt of Compound 1), in combination with tafasitamab, obinutuzumab, or tazemetostat, to patient having NHL. In another embodiment, provided herein are methods for achieving an increase in progression-free survival in a patient, comprising administering an effective amount of a compound described herein, e.g., Compound 1, or an enantiomer, mixture of enantiomers, tautomer, isotopolog, or pharmaceutically acceptable salt thereof (e.g., a hydrochloride salt of Compound 1), in combination with tafasitamab, obinutuzumab, or tazemetostat, to patient having NHL. In another embodiment, provided herein are methods for achieving an increase in event-free survival in a patient, comprising administering an effective amount of a compound described herein, e.g., Compound 1, or an enantiomer, mixture of enantiomers, tautomer, isotopolog, or pharmaceutically acceptable salt thereof (e.g., a hydrochloride salt of Compound 1), in combination with tafasitamab, obinutuzumab, or tazemetostat, to patient having NHL. In another embodiment, provided herein are methods for achieving an increase in time to progression in a patient, comprising administering an effective amount of a compound described herein, e.g., Compound 1, or an enantiomer, mixture of enantiomers, tautomer, isotopolog, or pharmaceutically acceptable salt thereof (e.g., a hydrochloride salt of Compound 1), in combination with tafasitamab, obinutuzumab, or tazemetostat, to patient having NHL. In another embodiment, provided herein are methods for achieving an increase in disease-free survival in a patient, comprising administering an effective amount of a compound described herein, e.g., Compound 1, or an enantiomer, mixture of enantiomers, tautomer, isotopolog, or pharmaceutically acceptable salt thereof (e.g., a hydrochloride salt of Compound 1), in combination with tafasitamab, obinutuzumab, or tazemetostat, to patient having NHL.

### Pharmaceutical Compositions and Routes of Administration

The compound provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds in the pharmaceutical composition may be at a level that will exercise the desired effect; about 0.001 mg/kg of a subject's body weight to about 1 mg/kg of a subject's body weight in unit dosage for both oral and parenteral administration.

A compound provided herein can be administered orally. In one embodiment, when administered orally, a compound provided herein is administered with a meal and water. In another embodiment, the compound provided herein is dispersed in water or juice (e.g., apple juice or orange juice) and administered orally as a solution or a suspension. In one embodiment, a compound provided herein is administered when the subject is fed. In one embodiment, a compound provided herein is administered when the subject is fed with high-fat and/or high-calorie food. In one embodiment, a compound provided herein is administered when the subject is fed with FDA-standard high-fat high-calorie breakfast. In one embodiment, a compound provided herein is administered when the subject is fasted. In one embodiment, a compound provided herein is administered after the subject has an at least 8-hour overnight fast. In one embodiment, a compound provided herein is administered with or without food.

The compound provided herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a compound provided herein without an additional carrier, excipient or vehicle. In another embodiment, provided herein are compositions comprising an effective amount of a compound provided herein and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts. In general, all of the compositions are prepared according to known methods in pharmaceutical chemistry. Capsules can be prepared by mixing a compound provided herein with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound provided herein as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound provided herein can be delayed or prolonged by proper formulation. For example, a slowly soluble pellet of the compound provided herein can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound provided herein in oily or emulsified vehicles that allow it to disperse slowly in the serum.

Certain pharmaceutical compositions and formulations of Compound 1 are described in U.S. patent application Ser. No. 17/075,447, the entirety of which is incorporated herein by reference.

The methods provided herein encompass treating a patient regardless of patient's age. In some embodiments, the subject is 18 years or older. In other embodiments, the subject is more than 18, 25, 35, 40, 45, 50, 55, 60, 65, or 70 years old. In other embodiments, the subject is less than 65 years old. In other embodiments, the subject is more than 65 years old.

Depending on the state of the disease to be treated and the subject's condition, Compound 1, or an enantiomer, mixture of enantiomers, tautomer, isotopolog, or pharmaceutically acceptable salt thereof, may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. Compound 1, or an enantiomer, mixture of enantiomers, tautomer, isotopolog, or pharmaceutically acceptable salt thereof, may be formulated, alone or together, in suitable dosage unit with pharmaceutically acceptable excipients, carriers, adjuvants and vehicles, appropriate for each route of administration.

In one embodiment, Compound 1, or an enantiomer, mixture of enantiomers, tautomer, isotopolog, or pharmaceutically acceptable salt thereof (e.g., a hydrochloride salt of Compound 1), is administered orally. In another embodiment, the compound of Compound 1, or an enantiomer, mixture of enantiomers, tautomer, isotopolog, or pharmaceutically acceptable salt thereof (e.g., a hydrochloride salt of Compound 1), is administered parenterally. In yet another embodiment, the compound of Compound 1, or an enantiomer, mixture of enantiomers, tautomer, isotopolog, or pharmaceutically acceptable salt thereof (e.g., a hydrochloride salt of Compound 1), is administered intravenously.

Compound 1, or an enantiomer, mixture of enantiomers, tautomer, isotopolog, or pharmaceutically acceptable salt thereof (e.g., a hydrochloride salt of Compound 1), can be delivered as a single dose such as, e.g., a single bolus injection, or oral capsules, tablets or pills; or over time, such as, e.g., continuous infusion over time or divided bolus doses over time. The compounds as described herein can be administered repeatedly if necessary, for example, until the patient experiences stable disease or regression, or until the patient experiences disease progression or unacceptable toxicity.

Compound 1, or an enantiomer, mixture of enantiomers, tautomer, isotopolog, or pharmaceutically acceptable salt thereof (e.g., a hydrochloride salt of Compound 1), can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as Compound 1, or an enantiomer, mixture of enantiomers, tautomer, isotopolog, or pharmaceutically acceptable salt thereof (e.g., a hydrochloride salt of Compound 1), is administered once or more than once each day, for example, for a period of time. The term “continuous” is intended to mean that a therapeutic compound, such as Compound 1, or an enantiomer, mixture of enantiomers, tautomer, isotopolog, or pharmaceutically acceptable salt thereof (e.g., a hydrochloride salt of Compound 1), is administered daily for an uninterrupted period of at least 7 days to 52 weeks. The term “intermittent” or “intermittently” as used herein is intended to mean stopping and starting at either regular or irregular intervals. For example, intermittent administration of Compound 1, or an enantiomer, mixture of enantiomers, tautomer, isotopolog, or pharmaceutically acceptable salt thereof (e.g., a hydrochloride salt of Compound 1), is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as Compound 1, or an enantiomer, mixture of enantiomers, tautomer, isotopolog, or pharmaceutically acceptable salt thereof (e.g., a hydrochloride salt of Compound 1), is administered daily or continuously but with a rest period.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, Compound 1, or an enantiomer, mixture of enantiomers, tautomer, isotopolog, or pharmaceutically acceptable salt thereof (e.g., a hydrochloride salt of Compound 1), is administered once a day. In another embodiment, Compound 1, or an enantiomer, mixture of enantiomers, tautomer, isotopolog, or pharmaceutically acceptable salt thereof, is administered twice a day. In yet another embodiment, Compound 1, or an enantiomer, mixture of enantiomers, tautomer, isotopolog, or pharmaceutically acceptable salt thereof (e.g., a hydrochloride salt of Compound 1), is administered three times a day. In still another embodiment, Compound 1, or an enantiomer, mixture of enantiomers, tautomer, isotopolog, or pharmaceutically acceptable salt thereof (e.g., a hydrochloride salt of Compound 1), is administered four times a day.

In certain embodiments, the methods provided herein include an administration of a therapeutically effective amount of Compound 1, or a pharmaceutically acceptable salt thereof (e.g., a hydrochloride salt of Compound 1) in one or more 7-day treatment cycles. In another embodiment, the methods provided herein include an administration of a therapeutically effective amount of Compound 1, or a pharmaceutically acceptable salt thereof (e.g., a hydrochloride salt of Compound 1) on days 1 to 5 of a 7-day cycle.

In certain embodiments, the methods provided herein include an administration of a therapeutically effective amount of Compound 1, or a pharmaceutically acceptable salt thereof (e.g., a hydrochloride salt of Compound 1) in one or more 14-day treatment cycles. In another embodiment, the methods provided herein include an administration of a therapeutically effective amount of Compound 1, or a pharmaceutically acceptable salt thereof (e.g., a hydrochloride salt of Compound 1) on days 1 to 5 of a 14-day cycle. In another embodiment, the methods provided herein include an administration of a therapeutically effective amount of Compound 1, or a pharmaceutically acceptable salt thereof (e.g., a hydrochloride salt of Compound 1) on days 1 to 7 of a 14-day cycle. In another embodiment, the methods provided herein include an administration of a therapeutically effective amount of Compound 1, or a pharmaceutically acceptable salt thereof (e.g., a hydrochloride salt of Compound 1) on days 1 to 10 of a 14-day cycle.

In one embodiment, the methods provided herein include an administration of a therapeutically effective amount of Compound 1, or a pharmaceutically acceptable salt thereof (e.g., a hydrochloride salt of Compound 1) in one or more 28-day treatment cycles. In another embodiment, the methods provided herein include an administration of a therapeutically effective amount of Compound 1, or a pharmaceutically acceptable salt thereof (e.g., a hydrochloride salt of Compound 1) on days 1 to 14 of a 28-day cycle. In another embodiment, the methods provided herein include an administration of a therapeutically effective amount of Compound 1, or a pharmaceutically acceptable salt thereof (e.g., a hydrochloride salt of Compound 1) on days 1 to 21 of a 28-day cycle. In another embodiment, the methods provided herein include an administration of a therapeutically effective amount of Compound 1, or a pharmaceutically acceptable salt thereof (e.g., a hydrochloride salt of Compound 1) on days 1 to 5, days 8 to 12, days 15 to 19, and days 22 to 26 of a 28-day cycle. In another embodiment, the methods provided herein include an administration of a therapeutically effective amount of Compound 1, or a pharmaceutically acceptable salt thereof (e.g., a hydrochloride salt of Compound 1) on days 1 to 5 and days 15 to 19 of a 28-day cycle. In another embodiment, the methods provided herein include an administration of a therapeutically effective amount of Compound 1, or a pharmaceutically acceptable salt thereof (e.g., a hydrochloride salt of Compound 1) on days 1 to 7 and days 15 to 21 of a 28-day cycle. In another embodiment, the methods provided herein include an administration of a therapeutically effective amount of Compound 1, or a pharmaceutically acceptable salt thereof (e.g., a hydrochloride salt of Compound 1) on days 1 to 10 and days 15 to 24 of a 28-day cycle.

In one embodiment, Compound 1, or a pharmaceutically acceptable salt thereof (e.g., a hydrochloride salt of Compound 1) is administered once daily for 5 days followed by 2 days of rest. In one embodiment, Compound 1, or a pharmaceutically acceptable salt thereof (e.g., a hydrochloride salt of Compound 1) is administered once daily for 5 days followed by 9 days of rest. In one embodiment, Compound 1, or a pharmaceutically acceptable salt thereof (e.g., a hydrochloride salt of Compound 1) is administered once daily for 7 days followed by 7 days of rest. In one embodiment, Compound 1, or a pharmaceutically acceptable salt thereof (e.g., a hydrochloride salt of Compound 1) is administered once daily for 10 days followed by 4 days of rest. In one embodiment, Compound 1, or a pharmaceutically acceptable salt thereof (e.g., a hydrochloride salt of Compound 1) is administered once daily for 14 days followed by 14 days of rest. In one embodiment, Compound 1, or a pharmaceutically acceptable salt thereof (e.g., a hydrochloride salt of Compound 1) is administered once daily for 21 days followed by 7 days of rest.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 5 days followed by 2 days of rest, starting on day 1 of the first 28-day cycle; and (ii) administering tafasitamab on days 1, 4, 8, 15, 22 of the first 28-day cycle, days 1, 8, 15, 22 of the second and third 28-day cycles, and then days 1 and 15 of the subsequent 28-day cycle(s), wherein the NHL is relapsed or refractory DLBCL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 5 days followed by 9 days of rest, starting on day 1 of the first 28-day cycle; and (ii) administering tafasitamab on days 1, 4, 8, 15, 22 of the first 28-day cycle, days 1, 8, 15, 22 of the second and third 28-day cycles, and then days 1 and 15 of the subsequent 28-day cycle(s), wherein the NHL is relapsed or refractory DLBCL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 7 days followed by 7 days of rest, starting on day 1 of the first 28-day cycle; and (ii) administering tafasitamab on days 1, 4, 8, 15, 22 of the first 28-day cycle, days 1, 8, 15, 22 of the second and third 28-day cycles, and then days 1 and 15 of the subsequent 28-day cycle(s), wherein the NHL is relapsed or refractory DLBCL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 10 days followed by 4 days of rest, starting on day 1 of the first 28-day cycle; and (ii) administering tafasitamab on days 1, 4, 8, 15, 22 of the first 28-day cycle, days 1, 8, 15, 22 of the second and third 28-day cycles, and then days 1 and 15 of the subsequent 28-day cycle(s), wherein the NHL is relapsed or refractory DLBCL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 14 days followed by 14 days of rest, starting on day 1 of the first 28-day cycle; and (ii) administering tafasitamab on days 1, 4, 8, 15, 22 of the first 28-day cycle, days 1, 8, 15, 22 of the second and third 28-day cycles, and then days 1 and 15 of the subsequent 28-day cycle(s), wherein the NHL is relapsed or refractory DLBCL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 21 days followed by 7 days of rest, starting on day 1 of the first 28-day cycle; and (ii) administering tafasitamab on days 1, 4, 8, 15, 22 of the first 28-day cycle, days 1, 8, 15, 22 of the second and third 28-day cycles, and then days 1 and 15 of the subsequent 28-day cycle(s), wherein the NHL is relapsed or refractory DLBCL.

In one embodiment, the method provided herein comprises administering a compound provided herein (e.g., Compound 1, or a pharmaceutically acceptable salt thereof, e.g., a hydrochloride salt of Compound 1) in combination with tafasitamab for a maximum of 12 28-day cycles, and then administering tafasitamab as monotherapy until clinical significant disease progression.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 5 days followed by 2 days of rest, starting on day 1 of the first 28-day cycle; and (ii) administering obinutuzumab on days 1, 8 and 15 of the first 28-day cycle and day 1 of the second to the sixth 28-day cycles, wherein the NHL is relapsed or refractory FL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 5 days followed by 9 days of rest, starting on day 1 of the first 28-day cycle; and (ii) administering obinutuzumab on days 1, 8 and 15 of the first 28-day cycle and day 1 of the second to the sixth 28-day cycles, wherein the NHL is relapsed or refractory FL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 7 days followed by 7 days of rest, starting on day 1 of the first 28-day cycle; and (ii) administering obinutuzumab on days 1, 8 and 15 of the first 28-day cycle and day 1 of the second to the sixth 28-day cycles, wherein the NHL is relapsed or refractory FL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 10 days followed by 4 days of rest, starting on day 1 of the first 28-day cycle; and (ii) administering obinutuzumab on days 1, 8 and 15 of the first 28-day cycle and day 1 of the second to the sixth 28-day cycles, wherein the NHL is relapsed or refractory FL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 14 days followed by 14 days of rest, starting on day 1 of the first 28-day cycle; and (ii) administering obinutuzumab on days 1, 8 and 15 of the first 28-day cycle and day 1 of the second to the sixth 28-day cycles, wherein the NHL is relapsed or refractory FL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 21 days followed by 7 days of rest, starting on day 1 of the first 28-day cycle; and (ii) administering obinutuzumab on days 1, 8 and 15 of the first 28-day cycle and day 1 of the second to the sixth 28-day cycles, wherein the NHL is relapsed or refractory FL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 5 days followed by 2 days of rest, starting on day 1 of the first 28-day cycle; and (ii) administering tazemetostat twice daily starting on day 1 of the first 28-day cycle, wherein the NHL is relapsed or refractory DLBCL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 5 days followed by 9 days of rest, starting on day 1 of the first 28-day cycle; and (ii) administering tazemetostat twice daily starting on day 1 of the first 28-day cycle, wherein the NHL is relapsed or refractory DLBCL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 7 days followed by 7 days of rest, starting on day 1 of the first 28-day cycle; and (ii) administering tazemetostat twice daily starting on day 1 of the first 28-day cycle, wherein the NHL is relapsed or refractory DLBCL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 10 days followed by 4 days of rest, starting on day 1 of the first 28-day cycle; and (ii) administering tazemetostat twice daily starting on day 1 of the first 28-day cycle, wherein the NHL is relapsed or refractory DLBCL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 14 days followed by 14 days of rest, starting on day 1 of the first 28-day cycle; and (ii) administering tazemetostat twice daily starting on day 1 of the first 28-day cycle, wherein the NHL is relapsed or refractory DLBCL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 21 days followed by 7 days of rest, starting on day 1 of the first 28-day cycle; and (ii) administering tazemetostat twice daily starting on day 1 of the first 28-day cycle, wherein the NHL is relapsed or refractory DLBCL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 5 days followed by 2 days of rest, starting on day 1 of the first 28-day cycle; and (ii) administering tazemetostat twice daily starting on day 1 of the first 28-day cycle, wherein the NHL is relapsed or refractory FL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 5 days followed by 9 days of rest, starting on day 1 of the first 28-day cycle; and (ii) administering tazemetostat twice daily starting on day 1 of the first 28-day cycle, wherein the NHL is relapsed or refractory FL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 7 days followed by 7 days of rest, starting on day 1 of the first 28-day cycle; and (ii) administering tazemetostat twice daily starting on day 1 of the first 28-day cycle, wherein the NHL is relapsed or refractory FL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 10 days followed by 4 days of rest, starting on day 1 of the first 28-day cycle; and (ii) administering tazemetostat twice daily starting on day 1 of the first 28-day cycle, wherein the NHL is relapsed or refractory FL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 14 days followed by 14 days of rest, starting on day 1 of the first 28-day cycle; and (ii) administering tazemetostat twice daily starting on day 1 of the first 28-day cycle, wherein the NHL is relapsed or refractory FL.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 21 days followed by 7 days of rest, starting on day 1 of the first 28-day cycle; and (ii) administering tazemetostat twice daily starting on day 1 of the first 28-day cycle, wherein the NHL is relapsed or refractory FL.

Any treatment cycle described herein can be repeated for at least 1, 2, 3, 4, 5, 6, 7, 8, or more cycles. In certain instances, the treatment cycle as described herein includes from 1 to about 24 cycles, from about 2 to about 16 cycles, or from about 2 to about 4 cycles. In certain instances a treatment cycle as described herein includes from 1 to about 4 cycles. In some embodiments, a therapeutically effective amount of Compound 1, or a pharmaceutically acceptable salt thereof (e.g., a hydrochloride salt of Compound 1), and/or a second therapeutic agent provided herein (e.g., tafasitamab, obinutuzumab, or tazemetostat) is administered for 1 to 13 cycles of 28 days (e.g., about 1 year). In certain instances, the cycling therapy is not limited to the number of cycles, and the therapy is continued until disease progression. Cycles can in certain instances include varying the duration of administration periods and/or rest periods described herein.

### EXAMPLES

The following Examples are presented by way of illustration, not limitation.

### Example 1: Phase I Clinical Study

A Phase 1, multi-center, open-label study is conducted to assess the safety, pharmacokinetics, and preliminary efficacy of an Orally Available Small Molecule, Compound 1, alone and in combination with anti-lymphoma agents in subjects with relapsed or refractory non-Hodgkin lymphomas (R/R NHL).

Indication: relapsed or refractory diffuse large B-cell lymphoma (R/R DLBCL); Relapsed or refractory follicular lymphoma (R/R FL); relapsed or refractory primary central nervous system lymphoma (R/R PCNSL); relapsed or refractory mantle cell lymphoma (R/R MCL).

Objectives: A primary objective of the study is to determine the safety and tolerability of Compound 1 alone and in combination with rituximab, obinutuzumab, tafasitamab, or tazemetostat in subjects with R/R NHL. Another primary objective is to define the maximum tolerated dose (MTD) and/or the recommended Phase 2 dose (RP2D) of Compound 1 in subjects with R/R NHL.

The secondary objectives are to characterize the pharmacokinetics (PK) of Compound 1 and to provide information on the preliminary efficacy of Compound 1 alone and in combination with rituximab, obinutuzumab, tafasitamab, or tazemetostat in R/R NHL.

Study Design: This is an open-label, Phase 1, dose escalation (Part A) and dose expansion (Part B), first-in-human (FIH) clinical study of Compound 1 administered orally alone and in combination with rituximab, obinutuzumab, tafasitamab, or tazemetostat. Compound 1 is administered in the form of a hydrochloride salt. Compound 1 is given as a monotherapy in subjects with R/R NHL, which includes DLBCL (de novo or transformed), FL, MCL or PCNSL who have failed at least 2 lines of therapy (or who have received at least one prior line of standard therapy and are not eligible for any other therapy). The combination of Compound 1 and rituximab is tested in subjects with R/R DLBCL or R/R FL. Compound 1 is also tested as a combination with obinutuzumab in R/R FL subjects, as a combination with tafasitamab in R/R DLBCL subjects, and as a combination with tazemetostat in R/R DLBCL and R/R FL subjects.

The dose escalation (Part A) evaluates the safety and tolerability of escalating doses of Compound 1 in R/R DLBCL and R/R FL to determine the MTD of Compound 1 as a monotherapy. An accelerated titration design is utilized at the initial dose levels; afterward, a two-parameter Bayesian logistic regression model (BLRM) utilizing escalation with overdose control (EWOC) (Babb J, Rogatko A, Zacks S. Cancer phase I clinical trials: efficient dose escalation with overdose control. Stat Med 1998; 17(10):1103-20; Neuenschwander B, Branson M, Gsponer T. Critical aspects of the Bayesian approach to phase I cancer trials. Stat Med 2008; 27(13); 2420-39), helps guide Compound 1 dose escalation/de-escalation decisions.

Part B evaluates the safety and efficacy of Compound 1 administered at or below the MTD alone or in combination in selected expansion cohorts of up to approximately 20 evaluable subjects each in R/R DLBCL and R/R FL, MCL, or PCNSL, in order to determine the RP2D. In addition to testing Compound 1 monotherapy, combinations to be tested in Part B may include: Compound 1 in combination with rituximab in R/R DLBCL and R/R FL subjects, Compound 1 in combination with obinutuzumab in R/R FL subjects, Compound 1 in combination with tafasitamab in R/R DLBCL subjects, and Compound 1 in combination with tazemetostat in R/R DLBCL and R/R FL subjects.

Part B Cohort B evaluates the effects of food on the PK of Compound 1 in subset of approximately 10-12 evaluable subjects with R/R FL, MCL, and/or PCNSL. The subjects are randomized to two groups and be administered an oral dose of Compound 1 after consumption of a standard high-fat, high-calorie breakfast at Cycle 1 Day 1 in Group 1 or the last dosing day in the first dosing period (e.g., Cycle 1 Day 7 in the 7/14 dosing schedule) in Group 2. During the rest of the treatment period, the subjects are administered oral doses of Compound 1 in the fasted state. Data from this food effect cohort provides exploratory assessment of impact of food on PK profile of Compound 1 in the subjects.

Compound 1 is administered orally once daily (QD) on planned dosing days. Part B expansion cohorts may test different doses and/or schedules of Compound 1 based on the safety and tolerability determined in Part A. All treatments are administered in 28-day cycles until clinically significant disease progression, unacceptable toxicity, or subject/physician decision to withdraw.

In Part A, each subject receives the assigned dose of Compound 1 on Cycle 1 Day 1 and daily on planned dosing days thereafter. The starting dose/schedule of Compound 1 is 0.4 mg/day starting on Day 1 for 5 consecutive days followed by 2 days off study drug every 7 days (5/7-day schedule) in each 28-day cycle. If the starting dose/schedule is not tolerated, a lower dose or less intense schedule may be explored. Planned dosing cohorts for Compound 1 in Part A include 0.1 mg (does level −2), 0.2 mg (does level −1), 0.4 mg (does level 1), 0.6 mg (does level 2), 0.8 mg (does level 3), 1.2 mg (does level 4), and 1.6 mg (does level 5). Alternate dosing schedules based on review of available clinical safety, PK and PD data may be explored, including, e.g., starting on Day 1 for 7 consecutive days followed by 7 days off study drug every 14 days (7/14-day schedule) in each 28-day cycle; starting on Day 1 for 5 consecutive days followed by 9 days off study drug every 14 days (5/14-day schedule) in each 28-day cycle; starting on Day 1 for 14 consecutive days followed by 14 days off study drug every 28 days (14/28-day schedule) in each 28-day cycle; and starting on Day 1 for 21 consecutive days followed by 7 days off study drug every 28 days (21/28-day schedule) in each 28-day cycle.

In Part A, after the first dose is administered at any dose level, subjects in each dose level are observed for at least 28 days (Cycle 1, Days 1 to 28, dose limiting toxicity [DLT] assessment period) before the next higher dose level can begin. The first 2 subjects within any escalated dose level enter the DLT period at least one day apart.

Following completion of dose escalation (Part A), selected expansion cohorts of approximately 20 efficacy evaluable subjects per cohort receive Compound 1 alone or in combination with rituximab, obinutuzumab, tafasitamab or tazemetostat. Dose, schedule and treatment regimen(s) can be selected for cohort expansion. Expansion may occur at the MTD established in the Part A and/or at a lower dose, or an alternative tolerable dosing schedule, based on review of available safety, PK, and PD data. A dose of Compound 1 can be chosen to be tested in combination with rituximab, obinutuzumab, tafasitamab, or tazemetostat in Part B that is evaluated initially in 6 subjects (safety run-in) before Cohorts of about 20 total subjects each C through H can be opened. If the dose of Compound 1 is not tolerated in combination with rituximab, obinutuzumab, tafasitamab, or tazemetostat, a lower dose level of Compound 1 may be explored. After review of the available safety, PK and/or PD observed in at least 6 subjects (safety run-in), the dose/schedule for Cohorts C through H can be selected. A combination expansion cohort may open once data for 6 subjects treated for that given combination has been reviewed. Recommended doses may differ for each combination. One or more dosing regimens may be selected for cohort expansion.

In addition, further dose exploration with prophylactic use of Granulocyte colony-stimulating factor (G-CSF) may be performed in Part A. Two findings could trigger such investigation: if neutropenia (ie, prolonged severe neutropenia or febrile neutropenia) is found to be the main toxicity determining the MTD, and if analysis of PD response indicates that maximum effect has not been attained, supporting exploration of higher Compound 1 doses. Upon review of the totality of PK, PD, safety and efficacy data, the SRC may choose to open a cohort within Part A, after establishing the MTD without the use of prophylactic G-CSF in Cycle 1 to test whether optimal management of neutropenia may benefit from prophylactic G-CSF use in Cycle 1. One or more cohorts in Part B may open based on the MTD as determined without the use of prophylactic G-CSF while the dose exploration with use of prophylactic G-CSF in Part A continues based on review of data.

Subjects enrolled into a cohort selected to receive prophylactic growth factor support with G-CSF (i.e., filgrastim) in Cycle 1, receive filgrastim via subcutaneous or IV injection two times a week on non-dosing weeks at 5 mcg/kg/day (e.g., 300 mcg for a 60 kg subject given two times a week on weeks 2 and 4 on the 7/14-day schedule and weeks 3 and 4 on the 14/28-day schedule). A different schedule for G-CSF may be used in Cycle 1. After Cycle 1, dose and schedule of prophylactic G-CSF (e.g., filgrastim or pegfilgrastim) are determined and adjusted if needed by the Investigator according to observed response for each subject. Therapeutic use of G-CSF in Cycle 1 and beyond is at the discretion of the Investigator. Filgrastim may be administered in either an inpatient of outpatient setting according to the locally approved filgrastim prescribing information or local institutional practice.

The study is conducted in compliance with International Conference on Harmonisation (ICH) Good Clinical Practices (GCPs).

Study Population: Subjects (male or female), ≥18 years of age, with R/R NHL who have relapsed after, progressed on (or not been able to tolerate due to medical comorbidities or unacceptable toxicity) standard anticancer therapy, or for whom no other approved conventional therapy exists, are enrolled in the study.

This multicenter, open-label study enrolls approximately 205 subjects with R/R NHL. Approximately 45 subjects with R/R DLBCL or R/R FL are enrolled in Part A to determine the MTD of Compound 1 monotherapy. Part B further evaluates the safety and efficacy of Compound 1 administered at or below the MTD (alone or in combination with rituximab, obinutuzumab, tafasitamab, or tazemetostat) in selected expansion cohorts in subjects with R/R DLBCL, FL, MCL or PCNSL of up to approximately 20 evaluable subjects in each cohort (C through H) in order to determine the RP2D. Potential food effects on Compound 1 PK are also investigated in a subset of 10-12 subjects in Part B Cohort B. Approximately 160 total subjects are studied in Part B to evaluate the RP2D. Enrollment occurs in the United States (US), Europe, and/or Canada for the study. Additional sites may be added for Part B.

Inclusion Criteria: Subjects must satisfy the following criteria to be enrolled in the study:
1. Subject is ≥18 years of age at the time of signing the informed consent form (ICF).
2. Subject must understand and voluntarily sign an ICF prior to any study-related assessments/procedures being conducted.
3. Subject is willing and able to adhere to the study visit schedule and other protocol requirements.
4. Subject has a history of NHL (including DLBCL [i.e., DLBCL NOS and high-grade B-cell lymphoma with MYC and BLC2 and/or BCL6 rearrangements with DLBCL morphology], FL, MCL, and PCNSL) with relapsed or refractory disease according to one of the following definitions:
   a. For R/R DLBCL (de novo): following at least 2 prior lines of therapy (e.g., have received first-line combination chemotherapy regimen containing rituximab, anthracycline, an alkylating agent, and steroids and have received at least one additional [second line/salvage] treatment) OR have failed at least one prior line of standard therapy and are not eligible for SCT. All subjects must not be eligible for any other approved treatment for their underlying lymphoma. Subjects with only one prior line of standard therapy must be ineligible for autologous SCT at the time of enrollment. Documentation of SCT ineligibility is required and acceptable reasons are only:
      Comorbidity, defined as any condition including laboratory abnormalities or clinical symptoms such as cardiac insufficiency and severe pulmonary diseases that place the subject at an unacceptable risk if he/she underwent SCT
      Subject declined SCT
      Physician considered the subject's disease could not be adequately treated by autologous SCT for possible reasons such as active disease following salvage therapy (ie. chemo-insensitive disease)
      Insufficient CD34 cell collection.
   b. For R/R DLBCL (transformed lymphoma): following at least 2 prior lines of therapy. No more than one of the prior lines of therapy may be a treatment for lower grade lymphoma; at least one standard treatment regimen for DLBCL must have been received.
   c. For R/R DLBCL (de novo or transformed lymphoma) enrolling in a tafasitamab cohort in Part B (ie, Compound 1+tafasitamab): following one to 3 systemic regimens (with at least one anti-CD20 therapy), who are not candidates for high-dose chemotherapy and subsequent autologous SCT. For subjects previously treated with CD19 CAR T therapy, a biopsy demonstrating CD19 positivity post CAR T therapy (ie, at relapse or if no response to CAR T) is required before enrollment in a tafasitamab cohort.
   d. For R/R FL: following at least 2 prior lines of therapy and meet treatment criteria at the time of enrollment based on investigator's assessment (e.g., according to Groupe d'Etude des Lymphomes Folliculaires [GELF] criteria [National Comprehensive Cancer Network. NCCN Clinical Practice Guidelines in Oncology: B-cell Lymphomas; V.2.2018. 2018 Feb. 26; V.2. Available from: https://www.nccn.org/professionals/physician_gls/pdf/b-cell.pdf]). Subjects with Grade 1, 2, 3a or 3b are eligible; subjects with Grade 1, 2, or 3a will be allocated to FL cohorts in Part B while subjects with Grade 3b will be enrolled into DLBCL cohorts in Part B. For subjects with FL Grade 3b enrolling in a tafasitamab cohort in Part B, following one to 3 systemic regimens (with at least one anti-CD20 therapy), who are not candidates for high-dose chemotherapy and subsequent autologous SCT. For subjects previously treated with CD19 CAR-T therapy, a recent (<1 month) biopsy demonstrating CD19+ relapse is required before enrollment in a tafasitamab cohort.
   e. For R/R MCL in Part B: following at least 2 prior lines of therapy. Subject must have progressed on or been refractory to at least one BTK inhibitor containing regimen (e.g., ibrutinib or acalabrutinib)
   f. For R/R PCNSL in Part B: following at least 2 prior lines of therapy. In addition, subjects with PCNSL must meet the following criteria: their neurological symptoms are stable (subjects taking glucocorticoids must be on a stable dose for 7 days prior to Day 1).
5. Subjects must have measurable disease:
   a. Defined by at least one fluorodeoxyglucose (FDG)-avid lesion for FDG-avid disease and one bi-dimensionally measurable disease on cross sectional imaging by computed tomography (CT) or magnetic resonance imaging (MRI) with at least one lesion >1.5 cm in the transverse diameter, as defined by the Lugano classification of NHL (Cheson B D, Fisher R I, Barrington S F, Cavalli F, Schwartz L H, Zucca E, et al., J Clin Oncol. 2014; 32(27):3059-3068). Measurable disease cannot be previously irradiated.
   b. PCNSL subjects in Part B must have disease that is objectively measurable by International Workshop to Standardize Baseline Evaluation and Response Criteria in Primary CNS Lymphoma (Abrey LE, et al., JCO: 2005, (23): 5034-5043), cerebrospinal fluid (CSF) cytology (in case of leptomeningeal only disease), or vitreal aspiration cytology and/or retinal photographs (in case of ocular lymphoma if clinically indicated).
6. Subject consents to retrieve formalin-fixed paraffin-embedded (FFPE) archival tumor tissue, either in tumor blocks or sectioned/mounted specimens, if collected within the last year and if using in place of the Screening biopsy for PD in Part A.
7. For subjects participating in Part A, subject consents to and has tumor accessible for tumor biopsy or FNA at Screening and FNA in Cycle 1; for Part B, subject consents to and has tumor accessible for paired tumor biopsies during Screening and Cycle 1.
8. Subject has an Eastern Cooperative Oncology Group (ECOG) performance status of 0, 1 or 2.
9. Subjects must have the following laboratory values:
   a. Absolute neutrophil count (ANC) ≥1.5×10⁹/L without growth factor support for 7 days (14 days if pegfilgrastim)
   b. Hemoglobin (Hgb) ≥8 g/dL
   c. Platelets (plt) ≥75×10⁹/L without transfusion for 7 days
   d. Aspartate aminotransferase/serum glutamic oxaloacetic transaminase (AST/SGOT) and alanine aminotransferase/serum glutamate pyruvic transaminase (ALT/SGPT) ≤2.5×upper limit of normal (ULN)
   e. Serum bilirubin ≤1.5×ULN except in cases of Gilberts Syndrome, then ≤2.0×ULN
   f. Estimated serum creatinine clearance of ≥60 mL/min using the Cockcroft-Gault equation or directly determined from the 24-hour urine collection method
   g. International normalized ratio (INR) <1.5×ULN and partial thromboplastin time (aPTT) <1.5×ULN (for subjects not receiving therapy). Note: Subjects receiving therapy for a thromboemboloic event that occurred >3 months prior to enrollment are eligible as long as they are on a stable regimen of anticoagulation with warfarin, low-molecular weight heparin or other approved therapeutic anticoagulation or antiplatelet regimen.
10. Subjects must agree not to donate blood while receiving Compound 1, during dose interruptions and for at least 28 days following the last dose of Compound 1.
11. Females of childbearing potential (FCBP) must adhere to Pregnancy Prevention Plan requirements, including:
   a. Either commit to true abstinence from heterosexual contact (which must be reviewed on a monthly basis and source documented) or agree to use, and be able to comply with, at least 2 effective contraceptive methods (oral, injectable, or implantable hormonal contraceptive; tubal ligation; intra-uterine device; barrier contraceptive with spermicide; or vasectomized partner), one of which must be barrier, from signing the ICF, at least 28 days before starting Compound 1, throughout the study, and for up to 28 days following the last dose of Compound 1, up to one year following the last dose of rituximab, up to 6 months following the last dose of tazemetostat, up to 18 months following the last dose of obinutuzumab, up to 3 months after the last dose of tafasitamab; and
   b. Have 2 negative pregnancy tests as verified by the Investigator prior to starting Compound 1: a negative serum pregnancy test (sensitivity of at least 25 mIU/mL) at Screening (between 10 to 14 days prior to Cycle 1 Day 1) and a negative serum or urine pregnancy test (Investigator's discretion) within 24 hours prior to Cycle 1 Day 1 of study treatment (note that the Screening serum pregnancy test can be used as the test prior to Day 1 study treatment if it is performed within the prior 24 hours).
   c. Avoid conceiving for 28 days after the last dose of Compound 1.
   d. Agree to ongoing pregnancy testing during the course of the study, and after the end of study treatment. This applies even if the subject practices true abstinence* from heterosexual contact.
   e. Agree to refrain from donating ova while on Compound 1 for 30 days after its discontinuation.
   f. Agree to abstain from breastfeeding or providing breast milk while on Compound 1 and for 28 days after its discontinuation.
12. Males must adhere to Pregnancy Prevention Plan requirements including the practice of true abstinence (which must be reviewed on a monthly basis) or agree to use a condom (a latex condom is recommended) during sexual contact with a pregnant female or a FCBP and will avoid conceiving from the date of signing the ICF, while participating in the study, during dose interruptions, and for at least 90 days following Compound 1 discontinuation, even if he has undergone a successful vasectomy. Males must agree to refrain from donating semen or sperm while on Compound 1 and for 90 days after its discontinuation. Males with female partners of reproductive potential must use effective contraception during treatment with tazemetostat and for 3 months after the final dose.
13. For subjects enrolling in food-effect subset of Cohort B in Part B: (1) Subject must agree and be willing to consume a standard high-fat, high-calorie meal; (2) Subject must be willing to refrain from caffeine or xanthene-containing products (coffee, tea, cola, chocolate, etc.) for 24 hours prior to dosing with food.

Exclusion Criteria: The presence of any of the following will exclude a subject from enrollment:
1. Subject has any significant medical condition, laboratory abnormality, or psychiatric illness that would prevent the subject from participating in the study.
2. Subject has any condition, including active or uncontrolled infection, or the presence of laboratory abnormalities, which places the subject at unacceptable risk if he/she were to participate in the study.
3. Subject has any condition that confounds the ability to interpret data from the study.
4. Subject has life expectancy ≤2 months.
5. Subjects who have aggressive lymphoma relapse requiring immediate cytoreductive therapy to avoid potential life-threatening consequences (e.g., due to tumor location).
6. Subject has received prior systemic anti-cancer treatment (approved or investigational) ≤5 half-lives or 4 weeks prior to starting Compound 1, whichever is shorter.
   a. Subjects who have had prior tafasitamab are excluded from enrolling in a tafasitamab cohort in Part B
   b. Subjects who have had prior tazemetostat are excluded from enrolling in a tazemetostat cohort in Part B
7. Subject has received prior CAR-T or other T-cell targeting treatment (approved or investigational) ≤4 weeks prior to starting Compound 1.
8. Subject has received prior therapy with CRBN-modulating drug (e.g., lenalidomide, avadomide/CC-122, pomalidomide) ≤4 weeks prior to starting Compound 1.
9. Subject is a pregnant or nursing female or intends to become pregnant during participation in the study.
10. Subject has symptomatic CNS involvement of disease (does not apply to PCNSL subjects in Part B).
11. Persistent diarrhea or malabsorption≥Grade 2 National Cancer Institute (NCI) Common Terminology Criteria for Adverse Events (CTCAE), despite medical management
12. Peripheral neuropathy≥NCI CTCAE Grade 2.
13. Subject is on chronic systemic immunosuppressive therapy or corticosteroids (e.g., prednisone or equivalent not to exceed 10 mg per day within the last 14 days) or subjects with clinically significant graft-versus-host disease (GVHD).
   a. Stable use of inhaled corticosteroids is allowed
   b. The use of topical steroids for ongoing skin or ocular GVHD is permitted
   c. In Part B, PCNSL subjects taking glucocorticoids are allowed but must be on a stable dose for 7 days prior to Cycle 1 Day 1
14. Subject has impaired cardiac function or clinically significant cardiac diseases, including any of the following:
   a. Left ventricular ejection fraction (LVEF) <45% as determined by multigated acquisition scan (MUGA) or echocardiogram (ECHO)
   b. Complete left bundle branch or bifascicular block
   c. Congenital long QT syndrome
   d. Persistent or clinically meaningful ventricular arrhythmias
   e. QTcF ≥470 msec on Screening electrocardiogram (ECG; mean of triplicate recordings)
   f. Unstable angina pectoris or myocardial infarction ≤3 months prior to starting 15. Subject had prior autologous SCT ≤3 months prior to starting Compound 1. If subject had prior autologous SCT >3 months prior to the start of Compound 1, any treatment-related toxicity is unresolved (Grade>1).
16. Subject had prior allogeneic SCT with either standard or reduced intensity conditioning ≤6 months prior to starting Compound 1. If subject had prior allogeneic SCT>6 months prior to the start of Compound 1, any treatment-related toxicity is unresolved (Grade>1).
17. Subject had major surgery ≤2 weeks prior to starting Compound 1. Subjects must have recovered from any clinically significant effects of recent surgery
18. Prior radiotherapy within one month prior to starting Compound 1.
19. Subject has known human immunodeficiency virus (HIV) infection.
20. Subject has known chronic active hepatitis B or C virus (HBV/HCV) infection.
21. Subject has a history of concurrent second cancers requiring active, ongoing systemic treatment.
22. Concurrent administration of strong CYP3A4/5 modulators.
23. Concurrent administration of strong or moderate CYP3A inducers for subjects enrolling on cohorts containing tazemetostat.

24. Previous SARS-COV-2 infection within 10 days for mild or asymptomatic infections or 20 days for severe/critical illness prior to starting Compound 1 (i.e., C1D1). Acute symptoms must have resolved and based on investigator assessment in consultation with the medical monitor, there are no sequelae that would place the participant at a higher risk of receiving study treatment.
25. Previous SARS-COV-2 vaccine within 14 days prior to starting Compound 1 (i.e., C1D1). For vaccines requiring more than one dose, the full series (e.g., both doses of a two-dose series) should be completed prior to enrollment when feasible and when a delay in enrollment would not put the study subject at risk.

Length of Study: The total study duration is expected to be approximately 5 to 6 years. Approximately 32 months are required to enroll and evaluate subjects in the dose escalation portion of the study (Part A). Approximately 13 to 19 months are required to enroll subjects in the Part B portion of the study. Completion of active treatment and post-treatment follow-up is expected to take an additional 12 to 24 months. The End of Trial is defined as either the date of the last visit of the last subject to complete the post-treatment follow-up, or the date of receipt of the last data point from the last subject that is required for primary, secondary and/or exploratory analysis, as pre-specified in the protocol, whichever is the later date.

Study Treatments: Subjects are assigned to a dose level and cohort by the Sponsor based on the subject's eligibility and slot availability. Subjects assigned to Dose Levels and single agent Cohorts (e.g., Cohorts A and B) in Part B receive Compound 1 as a monotherapy. Potential food effect on Compound 1 PK is also investigated in a subset of 10-12 subjects in Part B Cohort B. Subjects assigned to Part B combination Cohorts C through H receive Compound 1 in combination with rituximab, obinutuzumab, tafasitamab, or tazemetostat.

For subjects receiving rituximab in combination with Compound 1 in Part B, rituximab is administered (per package insert and institutional standard practice) on planned dosing days at the fixed dose of 375 mg/m2 for a maximum of 8 infusions. In Cycle 1, rituximab is given on Days 1, 8, 15, and 22; in Cycles 2-5, rituximab is given on Day 1 of each cycle or until clinically significant disease progression.

For subjects receiving obinutuzumab in combination with Compound 1 in Part B, obinutuzumab is administered (per package insert and institutional standard practice) on planned dosing days at the fixed dose of 1,000 mg. In Cycle 1, obinutuzumab is given on Days 1, 8, and 15; in Cycles 2-6, obinutuzumab is given on Day 1 of each cycle or until clinically significant disease progression.

For subjects receiving tafasitamab in combination with Compound 1 in Part B, tafasitamab is administered (per package insert and institutional standard practice) on planned dosing days at 12 mg/kg. In Cycle 1, tafasitamab is given on Days 1, 4, 8, 15 and 22; in Cycles 2 and 3, tafasitamab is given on Days 1, 8, 15 and 22, thereafter (Cycles 4+), tafasitamab is given once every other week (Q2W) on Days 1 and 15. For these subjects, Compound 1 is administered in combination with tafasitamab for a maximum of 12 cycles and then subjects continue tafasitamab monotherapy until clinically significant disease progression up to a maximum of 2 years total treatment duration.

For subjects receiving tazemetostat (TAZVERIK) in combination with Compound 1 in Part B, 800 mg tazemetostat is taken orally twice daily with or without food until clinically significant disease progression up to a maximum of 2 years total treatment duration.

Study treatments are discontinued after a total of 2 years and may also be discontinued if there is evidence of clinically significant disease progression, unacceptable toxicity, or subject/physician decision to withdraw. Subjects may discontinue the combination study treatment (ie, rituximab, obinutuzumab, tafasitamab or tazemetostat) if it is not tolerated and continue to receive Compound 1. Subjects may continue to receive study drugs beyond disease progression at the discretion of the Investigator in consultation with the Celgene Medical Monitor.

Overview of Key Efficacy Assessments: The primary efficacy variable is tumor response rate. Tumor responses are determined by the Investigator. For NHL, the International Workshop Criteria for Malignant Lymphoma (Cheson B D, et al., J Clin Oncol. 2014; 32(27):3059-3068) and the Deauville Criteria for fluorodeoxyglucose-positron emission tomography (FDG-PET) scan interpretation (Itti E, et al., Eur J Nucl Med Mol Imaging. 2013 September; 40(9):1312-20; Meignan M, et al., Leuk Lymphoma. 2014 January; 55(1):31-37) are used for efficacy assessment (“Lugano criteria”). Other response criteria are used as appropriate, including the International Workshop to Standardize Baseline Evaluation and Response Criteria in Primary CNS Lymphoma (Abrey L E, et al., JCO: 2005, (23): 5034-5043) for PCNSL. Efficacy variables to be analyzed include tumor response at the end of treatment, the proportion of subjects alive and progression-free, and duration of response.

Efficacy assessments include: clinical findings (e.g., physical examination, constitutional symptoms), contrast enhanced computed tomography (CT) scans where appropriate, FDG-PET/CT scans where appropriate, bone marrow examination (biopsy and aspiration) where appropriate, and magnetic resonance imaging (MRI) where appropriate.

Subjects are evaluated for efficacy at the ends of Cycles 2, 4, and 6; and then every 3 cycles thereafter until End of Treatment (EOT) using the same modalities used at Screening. All treated subjects are included in the efficacy analyses.

A descriptive analysis of evidence of antitumor activity is provided based on clinical, laboratory, and radiographic assessments, which includes assessment of target lesions, non-target lesions, new lesions and overall response.

The efficacy variable of focus for Part A is objective response rate (ORR). Additional efficacy variables to be analyzed include time to response, duration of response, progression-free survival (PFS) and overall survival (OS).

Efficacy variables mature when the last subject in each cohort has withdrawn from the study or completed one year of treatment.

Secondary and exploratory endpoints include evaluation of Compound 1 PD and predictive biomarkers in blood and/or tumor, and exploration of PK, PD, toxicity, and activity relationships.

Overview of Key Safety Assessments: Safety assessments include: monitoring for adverse events (AEs), physical examination, vital signs/weight, Eastern Cooperative Oncology Group (ECOG) performance status, safety laboratory assessments (including hematology and clinical chemistry, coagulation studies, and urinalysis), cardiac monitoring including 12-lead electrocardiograms (ECGs) and left ventricular ejection fraction (LVEF) assessments, concomitant medications, procedures, and therapies, and pregnancy testing (for females of child bearing potential [FCBP]).

Overview of Pharmacokinetic Assessments: The PK profiles of Compound 1 is determined from serial blood collections.

A number of references have been cited, the disclosures of which are incorporated herein by reference in their entirety.

The embodiments described above are intended to be merely exemplary, and those skilled in the art will recognize, or are able to ascertain using no more than routine experimentation, numerous equivalents of specific compounds, materials, and procedures. All such equivalents are considered to be within the scope of the invention and are encompassed by the appended claims.

What is claimed is:

1. A method of treating non-Hodgkin lymphoma (NHL), comprising administering to a subject in need thereof a therapeutically effective amount of a compound of Formula (I): 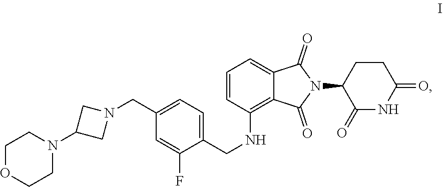 [SMILES: O=C1CC[C@H](N2C(=O)c3cccc(NCc4ccc(CN5CC(N6CCOCC6)C5)cc4F)c3C2=O)C(=O)N1]
or an enantiomer, mixture of enantiomers, tautomer, isotopolog, or pharmaceutically acceptable salt thereof, in combination with a second therapeutic agent, wherein the second therapeutic agent is tafasitamab, obinutuzumab, or tazemetostat.

2. The method of claim 1, provided that when the second therapeutic agent is tazemetostat, the NHL is follicular lymphoma (FL), primary central nervous system lymphoma (PCNSL), or mantle cell lymphoma (MCL).

3. The method of claim 1, wherein the NHL is diffuse large B-cell lymphoma (DLBCL), follicular lymphoma (FL), mantle cell lymphoma (MCL), or primary central nervous system lymphoma (PCNSL).

4. The method of claim 3, wherein the NHL is diffuse large B-cell lymphoma (DLBCL).

5. The method of claim 3, wherein the NHL is follicular lymphoma (FL).

6. The method of claim 3, wherein the NHL is mantle cell lymphoma (MCL).

7. The method of claim 3, wherein the NHL is primary central nervous system lymphoma (PCNSL).

8. The method of any one of claims 1 to 7, wherein the NHL is relapsed or refractory NHL.

9. The method of claim 8, wherein the NHL is relapsed or refractory DLBCL.

10. The method of claim 8, wherein the NHL is relapsed or refractory FL.

11. The method of claim 8, wherein the NHL is relapsed or refractory MCL.

12. The method of claim 8, wherein the NHL is relapsed or refractory PCNSL.

13. The method of any one of claims 8 to 12, wherein the subject has received at least one prior line of therapy.

14. The method of any one of claims 8 to 12, wherein the subject has received at least two prior lines of therapy.

15. The method of any one of claims 8 to 12, wherein the subject has received one to three systemic regimens, and wherein at least one of the systemic regimens is an anti-CD20 therapy.

16. The method of any one of claims 1 to 7, wherein the NHL is newly diagnosed.

17. The method of any one of claims 1 to 16, wherein the NHL is characterized by the presence of an Enhancer of Zeste Homolog 2 (EZH2) mutation.

18. The method of any one of claims 1 to 17, wherein the second therapeutic agent is tafasitamab.

19. The method of claim 18, wherein tafasitamab is administered via intravenous infusion.

20. The method of claim 18 or 19, wherein tafasitamab is administered in an amount of about 12 mg/kg of the body weight of the subject.

21. The method of any one of claims 18 to 20, wherein tafasitamab is administered on days 1, 4, 8, 15, and 22 of the first 28-day cycle, days 1, 8, 15, and 22 of the second and third 28-day cycles, and days 1 and 15 of the subsequent 28-day cycle(s).

22. The method of any one of claims 1 to 17, wherein the second therapeutic agent is obinutuzumab.

23. The method of claim 22, wherein obinutuzumab is administered via intravenous infusion.

24. The method of claim 22 or 23, wherein obinutuzumab is administered in an amount of about 1000 mg.

25. The method of any one of claims 22 to 24, wherein obinutuzumab is administered on days 1, 8 and 15 of the first 28-day cycle and day 1 of the second to the sixth 28-day cycles.

26. The method of any one of claims 1 to 17, wherein the second therapeutic agent is tazemetostat.

27. The method of claim 26, wherein tazemetostat is administered orally.

28. The method of claim 26 or 27, wherein tazemetostat is administered with food.

29. The method of claim 26 or 27, wherein tazemetostat is administered without food.

30. The method of any one of claims 26 to 29, wherein tazemetostat is administered in an amount of about 400 mg, about 600 mg or about 800 mg.

31. The method of any one of claims 26 to 30, wherein tazemetostat is administered twice daily.

32. The method of any one of claims 1 to 31, wherein a compound of Formula (I), or a pharmaceutically acceptable salt thereof, is administered.

33. The method of claim 32, wherein a hydrochloride salt of a compound of Formula (I) is administered.

34. The method of any one of claims 1 to 33, wherein the compound is administered orally.

35. The method of claim 34, wherein the compound is administered when the subject is fed.

36. The method of claim 34, wherein the compound is administered when the subject is fasted.

37. The method of any one of claims 1 to 36, wherein the compound is administered
(a) once daily for 5 days followed by 2 days of rest;
(b) once daily for 5 days followed by 9 days of rest;
(c) once daily for 7 days followed by 7 days of rest;
(d) once daily for 14 days followed by 14 days of rest; or
(e) once daily for 21 days followed by 7 days of rest.

38. The method of any one of claims 1 to 36, wherein the compound is administered on days 1 to 5 of a 7-day cycle, on days 1 to 5 of a 14-day cycle, on days 1 to 7 of a 14-day cycle, on days 1 to 10 of a 14-day cycle, on days 1 to 14 of a 28-day cycle, on days 1 to 21 of a 28-day cycle, on days 1 to 5, days 8 to 12, days 15 to 19, and days 22 to 26 of a 28-day cycle, on days 1 to 5 and days 15 to 19 of a 28-day cycle, on days 1 to 7 and days 15 to 21 of a 28-day cycle, or on days 1 to 10 and days 15 to 24 of a 28-day cycle.

39. The method of any one of claims 1 to 38, wherein the compound is administered in an amount of about 0.1 mg, about 0.2 mg, about 0.4 mg, about 0.6 mg, about 0.8 mg, about 1.2 mg or about 1.6 mg per day.

40. The method of claim 1, comprising (i) administering the compound in cycles of once daily for 5 days followed by 2 days of rest, starting on day 1 of the first 28-day cycle; and (ii) administering tafasitamab on days 1, 4, 8, 15, and 22 of the first 28-day cycle, days 1, 8, 15, and 22 of the second and third 28-day cycles, and days 1 and 15 of the subsequent 28-day cycle(s), wherein the NHL is relapsed or refractory DLBCL.

41. The method of claim 1, comprising (i) administering the compound in cycles of once daily for 5 days followed by 9 days of rest, starting on day 1 of the first 28-day cycle; and (ii) administering tafasitamab on days 1, 4, 8, 15, and 22 of the first 28-day cycle, days 1, 8, 15, and 22 of the second and third 28-day cycles, and days 1 and 15 of the subsequent 28-day cycle(s), wherein the NHL is relapsed or refractory DLBCL.

42. The method of claim 1, comprising (i) administering the compound in cycles of once daily for 7 days followed by 7 days of rest, starting on day 1 of the first 28-day cycle; and (ii) administering tafasitamab on days 1, 4, 8, 15, and 22 of the first 28-day cycle, days 1, 8, 15, and 22 of the second and third 28-day cycles, and days 1 and 15 of the subsequent 28-day cycle(s), wherein the NHL is relapsed or refractory DLBCL.

43. The method of claim 1, comprising (i) administering the compound in cycles of once daily for 14 days followed by 14 days of rest, starting on day 1 of the first 28-day cycle; and (ii) administering tafasitamab on days 1, 4, 8, 15, and 22 of the first 28-day cycle, days 1, 8, 15, and 22 of the second and third 28-day cycles, and days 1 and 15 of the subsequent 28-day cycle(s), and then once every two weeks, wherein the NHL is relapsed or refractory DLBCL.

44. The method of claim 1, comprising (i) administering the compound in cycles of once daily for 21 days followed by 7 days of rest, starting on day 1 of the first 28-day cycle; and (ii) administering tafasitamab on days 1, 4, 8, 15, and 22 of the first 28-day cycle, days 1, 8, 15, and 22 of the second and third 28-day cycles, and days 1 and 15 of the subsequent 28-day cycle(s), wherein the NHL is relapsed or refractory DLBCL.

45. The method of claim 1, comprising (i) administering the compound in cycles of once daily for 5 days followed by 2 days of rest, starting on day 1 of the first 28-day cycle; and (ii) administering obinutuzumab on days 1, 8 and 15 of the first 28-day cycle and day 1 of the second to the sixth 28-day cycles, wherein the NHL is relapsed or refractory FL.

46. The method of claim 1, comprising (i) administering the compound in cycles of once daily for 5 days followed by 9 days of rest, starting on day 1 of the first 28-day cycle; and (ii) administering obinutuzumab on days 1, 8 and 15 of the first 28-day cycle and day 1 of the second to the sixth 28-day cycles, wherein the NHL is relapsed or refractory FL.

47. The method of claim 1, comprising (i) administering the compound in cycles of once daily for 7 days followed by 7 days of rest, starting on day 1 of the first 28-day cycle; and (ii) administering obinutuzumab on days 1, 8 and 15 of the first 28-day cycle and day 1 of the second to the sixth 28-day cycles, wherein the NHL is relapsed or refractory FL.

48. The method of claim 1, comprising (i) administering the compound in cycles of once daily for 14 days followed by 14 days of rest, starting on day 1 of the first 28-day cycle; and (ii) administering obinutuzumab on days 1, 8 and 15 of the first 28-day cycle and day 1 of the second to the sixth 28-day cycles, wherein the NHL is relapsed or refractory FL.

49. The method of claim 1, comprising (i) administering the compound in cycles of once daily for 21 days followed by 7 days of rest, starting on day 1 of the first 28-day cycle; and (ii) administering obinutuzumab on days 1, 8 and 15 of the first 28-day cycle and day 1 of the second to the sixth 28-day cycles, wherein the NHL is relapsed or refractory FL.

50. The method of claim 1, comprising (i) administering the compound in cycles of once daily for 5 days followed by 2 days of rest, starting on day 1 of the first 28-day cycle; and (ii) administering tazemetostat twice daily starting on day 1 of the first 28-day cycle, wherein the NHL is relapsed or refractory DLBCL or relapsed or refractory FL.

51. The method of claim 1, comprising (i) administering the compound in cycles of once daily for 5 days followed by 9 days of rest, starting on day 1 of the first 28-day cycle; and (ii) administering tazemetostat twice daily starting on day 1 of the first 28-day cycle, wherein the NHL is relapsed or refractory DLBCL or relapsed or refractory FL.

52. The method of claim 1, comprising (i) administering the compound in cycles of once daily for 7 days followed by 7 days of rest, starting on day 1 of the first 28-day cycle; and (ii) administering tazemetostat twice daily starting on day 1 of the first 28-day cycle, wherein the NHL is relapsed or refractory DLBCL or relapsed or refractory FL.

53. The method of claim 1, comprising (i) administering the compound in cycles of once daily for 14 days followed by 14 days of rest, starting on day 1 of the first 28-day cycle; and (ii) administering tazemetostat twice daily starting on day 1 of the first 28-day cycle, wherein the NHL is relapsed or refractory DLBCL or relapsed or refractory FL.

54. The method of claim 1, comprising (i) administering the compound in cycles of once daily for 21 days followed by 7 days of rest, starting on day 1 of the first 28-day cycle; and (ii) administering tazemetostat twice daily starting on day 1 of the first 28-day cycle, wherein the NHL is relapsed or refractory DLBCL or relapsed or refractory FL.

55. The method of any one of claims 1 to 54, wherein the method further comprises administering to the subject granulocyte-colony stimulating factor (G-CSF) or granulocyte-macrophage colony stimulating factor (GM-CSF).

56. The method of claim 55, wherein the G-CSF is filgrastim or pegfilgrastim.

57. The method of claim 56, wherein filgrastim is administered in an amount of about 5 mcg/day/kg of the body weight of the subject.

58. The method of any one of claims 55 to 57, wherein the G-CSF is administered via subcutaneous or intravenous injection.

59. The method of any one of claims 55 to 58, wherein G-CSF is administered on days 15 to 28 of a 28-day cycle whereas the compound is administered on days 1 to 14 of the 28-day cycle; or the G-CSF is administered on days 8 to 14 and days 22 to 28 of a 28-day cycle whereas the compound is administered on days 1 to 7 and days 15 to 21 of the 28-day cycle.
